(19) United States
(12) Patent Application Publication
(10) Pub. No.: US 2025/0275926 A1
(43) Pub. Date: Sep. 4, 2025

(54) MULTILAYER FORMULATION COMPRISING MODIFIED TAPIOCA STARCH FOR CONTROLLED-RELEASE OF ACTIVE AGENTS

(71) Applicant: ACCESS BUSINESS GROUP INTERNATIONAL LLC, Ada, MI (US)

(72) Inventors: Rohit P. DUGAR, Grand Rapids, MI (US); Shyam RAMAKRISHNAN, Selangor (MY); Marines MATHIS, La Verne, CA (US); Jacob LIM, Tustin, CA (US); Anshuman MOHANTY, Balasore (IN); Aparna DAMLE, Bangalore (IN); Vijendra Kumar Kanaganamaradi BOMMEGOWDA, Pandavapura (IN); Ugaraj Raja GOUNDER, Karur (IN)

(21) Appl. No.: 18/858,027

(22) PCT Filed: Apr. 19, 2023
(86) PCT No.: PCT/US2023/019084

§ 371 Date: Oct. 18, 2024

Related U.S. Application Data
(60) Provisional application No. 63/332,714, filed on Apr. 20, 2022.

(51) Int. Cl. A61K 9/24; A61K 9/28; A61K 31/07; A61K 31/355; A61K 31/593; A61K 33/04; A61K 33/06; A61K 33/18; A61K 33/22; A61K 33/24; A61K 33/34; A61K 33/42

(52) U.S. Cl. CPC A61K 9/209; A61K 9/286; A61K 31/07; A61K 31/355; A61K 31/593; A61K 33/04; A61K 33/06; A61K 33/18; A61K 33/22; A61K 33/24; A61K 33/34; A61K 33/42

(57) ABSTRACT

Provided is a multilayer formulation. The multilayer formulation is useful for controlled-release and immediate-release of active agents after administration to a subject. The multilayer formulation comprises a first layer comprising a controlled-release composition, and a second layer disposed adjacent the first layer and comprising an immediate-release composition. The controlled-release composition of the first layer comprises a first active agent and a controlled-release excipient comprising a modified tapioca starch. The modified tapioca starch provides for controlled-release of the first active agent from the first layer. The immediate-release composition of the second layer comprises a second active agent and an excipient. The multilayer formulation can be in the form of a tablet, such as a bilayer tablet. The multilayer formulation may be administered orally to a subject to provide various types of active agents (e.g., vitamins, minerals, botanicals, etc.) at different locations of the subject's gastrointestinal (GI) tract.

### CROSS-REFERENCE TO RELATED APPLICATIONS

This application claims priority to and all benefits of U.S. Provisional Patent Application No. 63/332,714 filed on 20 Apr. 2022, the content of which is incorporated herein by reference.

### TECHNICAL FIELD

The present disclosure generally relates to a multilayer formulation for release of active agents. The present disclosure also relates to a method of manufacturing the multilayer formulation, a method of delivering active agents to a subject, and use of modified tapioca starch to modify the release rate of actives, e.g. in a subject's gastrointestinal (GI) tract after ingestion of a formulation. The multilayer formulation can be in the form for administration to a subject, e.g., a bilayer tablet.

### BACKGROUND OF THE INVENTION

Controlled-release of an active agent allows control of a subject's administration scheme of the active agent by reducing the amount of recommended daily intakes, improves patient compliance, and attenuates adverse events, e.g., related to high plasma peaks. Controlled-release oral compositions regulate the release of the incorporated active agent(s) over time and includes formulations with a controlled, a prolonged, a sustained, a delayed, a slow, and/or an extended-release, for accomplishing therapeutic or convenience objectives not offered by conventional oral dosage forms such as solutions or rapidly dissolving dosage forms.

Controlled-release of active agents from an oral composition (e.g., a tablet) may be accomplished by homogeneously embedding the active agents in a hydrophilic matrix, being a soluble, partially soluble or insoluble network of viscous, hydrophilic polymers, held together by physical or chemical entanglements, by ionic or crystalline interactions, by complex formation, by hydrogen bonds or van der Waals forces. The hydrophilic matrix swells upon contact with water, thereby creating a protective gel layer from which the active agents are slowly, gradually, or continuously released in time either by diffusion through the polymeric network, by erosion of the gel layer, by dissolution of the polymer, or by a combination of these release mechanisms.

In the dietary supplements industry, starch is used as a common excipient in dietary supplement products. Starches are obtained from various botanical sources, for example, corn, potato, cassava or tapioca, rice and wheat. Corn starch is mostly used when compared with other starches because of its well-known properties and availability of pharmaceutical grade raw materials in the market. Conventionally, native tapioca starch can be used as diluent, binder, and disintegrant in tablet and capsule formulations.

Tapioca starch is derived from the roots of cassava (Manihot esculenta Crantz). Starch is the major component of cassava root and can comprise up to 80% of the dried weight of the root. Cassava is abundantly available in the world, thus the supply of tapioca starch is sustainable and cheap. However, native tapioca starch does not provide oral compositions with suitable controlled-release of active agents within the oral composition that is desired by the subjects or their care-givers.

An adequate intake of vitamins and minerals is important for health and can be obtained through a healthy diet. In the absence of such a diet, dietary supplements may be useful sources of one or more of these micronutrients that otherwise might be consumed in less than recommended amounts. Supplemental vitamins and minerals may also be required when gastrointestinal absorption is impaired, where there are excessive losses, or increased requirements. Consequences of deficiency of one or more vitamins or minerals can be serious.

Many time release products on the market use high amounts of polymers to achieve their time release profile. For example, manufacturers have used hydrophilic hydrocolloid gelling polymers such as hydroxypropyl methylcellulose, hydroxypropyl cellulose, or Pullulan to formulate sustained release tablets or capsules. These polymers first form a gel when exposed to an aqueous environment of low pH thereby slowly diffusing the active medicament which is contained within the polymer matrix. When the gel enters a higher pH environment such as that found in the intestines, however, it dissolves resulting in a less controlled drug release. To provide better sustained release properties in higher pH environments, some pharmaceutical manufacturers use polymers which dissolve only at higher pHs, such as acrylic resins, acrylic latex dispersions, cellulose acetate phthalate, and hydroxypropyl methylcellulose phthalate, either alone or in combination with hydrophilic polymers.

Generally, these formulations are prepared by combining actives with a finely divided powder of the hydrophilic polymer, or the hydrophilic and water-insoluble polymers. These ingredients are mixed and granulated with water or an organic solvent and the granulation is dried. The dry granulation is then usually further blended with various pharmaceutical additives and compressed into tablets.

Although these types of formulations have been successfully used to manufacture dosage forms which demonstrate sustained release properties, these formulations generally do not have the desired release profile or serum concentration of actives over an extended period of time. Also, having polymers can be undesirable when trying to include mainly natural ingredients within a product.

Accordingly, there is an opportunity to provide formulations exhibiting controlled-release of active agents and methods relating to the same. Furthermore, other improved features and characteristics will become apparent from the subsequent summary and detailed description and the appended claims, taken in conjunction with the foregoing technical field and background.

### BRIEF SUMMARY OF THE INVENTION

A multilayer formulation is provided. The multilayer formulation is useful for controlled-release and immediate-release of active agents after administration of the multilayer formulation to a subject. The multilayer formulation comprises a first layer and a second layer disposed adjacent the first layer. Optionally, an outer coating covers the first and second layers.

The first layer comprises (and/or is formed from) a controlled-release composition. The controlled-release composition comprises a first active agent and a controlled-release excipient. The controlled-release excipient comprises a modified tapioca starch.

The controlled-release composition is adapted to release the first active agent in a modified way. In various embodiments, no greater than 50 wt. % of the first active agent is released within 1 hour of ingestion of the multilayer formulation by a subject, based on a total weight of the first active agent present in the controlled-release composition. In further or other embodiments, not less than 55 wt. % of the first active agent is released within 6 hours after ingestion of the multilayer formulation by the subject, based on a total weight of the first active agent present in the controlled-release composition. In certain embodiments, such released amounts are based on the total weight (or total amount) of the first active agent present in the multilayer formulation as a whole.

The second layer comprises (and/or is formed from) an immediate-release composition. The immediate-release composition is different from the controlled-release composition associated with the first layer.

The immediate-release composition comprises a second active agent and an excipient. The excipient of the immediate-release composition is different from the controlled-release excipient of the controlled-release composition, e.g., in at least one of makeup, type, amount, etc. The second active agent may be the same as or different from the first active agent. In various embodiments, the active agents are different, e.g., in at least one of makeup, type, amount, etc.

The immediate-release composition is adapted to release the second active agent in a manner different from that of first active agent release. In general, a percentage of total second active agent released from the multilayer formulation is greater than a percentage of total first active agent released from the multilayer formulation during the same period of time. In this way, the active agents may be released at different locations in a subject's gastrointestinal (GI) tract. For example, the second active agent is predominantly released “upstream” of the first active agent, although there may also be some overlap of actives released in the subject's GI tract.

In various embodiments, not less than 60 wt. % of the second active agent is released within 1 hour after ingestion of the multilayer formulation by a subject, based on a total weight of the second active agent present in the immediate-release composition. In certain embodiments, such released amounts is based on the total weight (or total amount) of the second active agent present in the multilayer formulation as a whole.

A method of manufacturing the multilayer formulation is also provided. The method comprises providing the controlled-release composition, and providing the immediate-release composition. The method further comprises forming the first layer from the controlled-release composition, and forming the second layer from the immediate-release composition. The method yet further comprises combining the first and second layers to form the multilayer formulation. The steps of forming and combining the layers may be separate or may occur simultaneously.

A method of delivering active agents to a subject is also provided. The method comprises providing the multilayer formulation. The method further comprising administering the multilayer formulation to the subject. For example, the multilayer formulation may be administered orally to the subject.

Use of a modified tapioca starch as a controlled-release excipient in a formulation for forming tablets is also provided. In various embodiments, the modified tapioca starch comprises a hydroxypropyl tapioca starch.

In various embodiments, this disclosure relates to a formulation design and development of a bilayer tablet, and more specifically, to a new formula design of a dual layer, dual release tablet where each layer has specific ingredients, including one or more vitamins, minerals, and/or botanicals (collectively, “nutrients” or “micronutrients”). As will be appreciated with reference to the description herein, the two layers have different dissolution release profiles, namely immediate release and extended release. In certain embodiments, immediate release refers to release of one or more nutrients within one hour of ingestion; whereas, extended release refers to about six hours. Specific vitamins and minerals for each layer can be selected based on the presence of preferential absorption sites throughout the GI tract. Likewise, specific botanicals can be selected based on preferential sites of release and delivery after ingestion of the bilayer tablet.

### BRIEF DESCRIPTION OF THE DRAWINGS

FIG. 1 is a cross-sectional view of an embodiment of a multilayer formulation, more specifically, a bilayer tablet;

FIG. 2 is a photograph of an embodiment of the bilayer tablet;

FIG. 3A is a perspective view of another embodiment of the bilayer tablet;

FIG. 3B is a cross-sectional view of another embodiment of the bilayer tablet;

FIG. 4A is a perspective view of another embodiment of a multilayer formulation, more specifically, a trilayer tablet;

FIG. 4B is a cross-sectional view of another embodiment of the trilayer tablet;

FIG. 4C is a cross-sectional view of another embodiment of the trilayer tablet;

FIG. 4D is a cross-sectional view of yet another embodiment of the trilayer tablet;

FIG. 5 is a simple diagram of a human gastrointestinal (GI) tract with various components and locations of absorbance;

FIG. 6 is a schematic of an embodiment of a manufacturing process for forming a bilayer tablet;

FIG. 7 is a graph showing the percent niacinamide release over time;

FIG. 8 is a graph showing the percent iron release over time;

FIG. 9 is a graph showing the percent riboflavin release over time; and

FIG. 10 is a graph showing the percent folic acid release over time.

### DETAILED DESCRIPTION OF THE INVENTION

Except in the Examples, or where otherwise expressly indicated, all numerical quantities in this description indicating amounts of material or conditions of reaction and/or use are to be understood as modified by the word “about” in describing the broadest scope of the disclosure. In various embodiments, the terms “about” and “approximately,” when referring to a specified, measurable value (such as a parameter, an amount, a temporal duration, and the like), is meant to encompass the specified value and variations of and from the specified value, such as variations of ±10% or less, optionally ±5% or less, optionally ±1% or less, or optionally ±0.1% or less of and from the specified value, insofar as such variations are appropriate to perform in the disclosed embodiments. Thus the value to which the modifier “about” or “approximately” refers is itself also specifically disclosed.

Unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It must also be noted that, as used in the specification and appended claims, the singular form “a,” “an,” and “the” comprise plural referents unless the context clearly indicates otherwise. For example, reference to a component in the singular is intended to comprise a plurality of components.

As used herein, an “embodiment” means that a particular feature, structure or characteristic is included in at least one or more manifestations, examples, or implementations of the invention. Furthermore, the particular features, structures or characteristics may be combined in any suitable manner, as would be apparent to a person skilled in the art. Combinations of features of different embodiments are all meant to be within the scope of the invention, without the need for explicitly describing every possible permutation by example. Thus, any of the claimed embodiments can be used in any combination.

As used herein, the term “weight percent” (and thus the associated abbreviation “wt. %”) typically refers to a percent by weight expressed in terms of a weight of dry matter. As such, it is to be appreciated that a wt. % can be calculated on a basis of a total weight of a composition, or calculated from a ratio between two or more components/parts of a mixture (e.g. a total weight of dry matter).

Throughout this disclosure, where patents, publications, or non-patent literature references are referenced, the disclosures of these documents in their entireties are hereby incorporated by reference into this disclosure to more fully describe the state of the art to which this disclosure pertains.

### Definitions

The following definitions are generally used to describe the various aspects and characteristics of the claimed formulations and processes.

USP General Chapter <1151> PHARMACEUTICAL DOSAGE FORMS states that there are two principal categories of drug release: immediate-release and modified-release. “Immediate-release” is observed when no deliberate effort has been made to modify the drug substance release profile. For example, capsules and tablets are considered immediate release even if a disintegrating agent or a lubricant has been used. “Modified-release” is a term used when the rate and/or time of release of the drug substance is altered as compared to what would be observed or anticipated for an immediate-release product. Two modified-release profiles, delayed-release and extended-release are recognized. The term “modified-release” is not used for official article titles. “Delayed-release” is used when a deliberate formulation achieves a delay in the release of the drug substance for some period of time after initial administration. For oral products, expressions such as “enteric-coated” or “gastro-resistant” also have been used where release of the drug substance is prevented in the gastric environment but promoted in the intestinal environment. However, the term “delayed-release” is used for official article titles. “Extended-release” is used when the deliberate formulation achieves prolongation of drug substance release compared to that observed or anticipated for an immediate-release dosage form. Expressions such as “prolonged-release”, “repeat-action”, “controlled-release”, “long-acting”, and “sustained-release” also have been used to describe such dosage forms. However, the term “extended-release” is used for official article titles.

In view of the above background, and as used herein, the term “immediate-release” generally means that the release of the components from the formulation is not delayed once at its intended location, e.g., the rapid break-up and delivery of a portion of the formulation in the upper-to mid-GI tract. In various embodiments, near-complete to complete release of the components may occur after about 1 hour or less.

In addition, the term “controlled-release” is generally intended to exclude immediate-release and to encompass modified-release, extended-release, pulsed-release, sustained-release, delayed-release, timed-release, variable-release and combinations thereof. Thus, each of these terms may be used interchangeably.

Moreover, the term “extended-release” generally denotes the duration of time between administration of the formulation and the release of the components from a particular layer. In various embodiments, near-complete to complete release of the components may occur after about 6 to about 8 hours, e.g., the rapid break-up and delivery of a portion of the formulation in the mid-to lower-GI tract.

The term “sufficient amounts” generally means that the amounts meet the currently accepted recommended dietary allowances and intakes for humans.

An “active” or “active agent” generally refers to any drug, medicine, phytochemical, or supplement, such as a vitamin, a mineral, a botanical supplement, or the like. The terms “active” and “active agent” may be used interchangeably.

The terms “composition” or “formulation” generally refer to a product that treats, improves, promotes, increases, manages, controls, maintains, optimizes, modifies, reduces, inhibits, or prevents a particular condition associated with a natural state, biological process or disease or disorder. The terms “composition” and “formulation” may be used interchangeably.

The terms “applying” and “administering” are generally defined as providing a composition to a subject via a route known in the art, including but not limited to topical, intravenous, intra-arterial, oral, parenteral, buccal, transdermal, rectal, intramuscular, subcutaneous, intraosseous, transmucosal, or intraperitoneal routes of administration. In general, the formulation of this disclosure is administered orally.

As used herein, the term “subject” or “individual” generally includes mammals to which a composition may be administered. Non-limiting examples of mammals include humans, non-human primates, livestock, rodents (including transgenic and non-transgenic mice) or the like. In various embodiments, the subject is a mammal, and in many embodiments, the subject is human. In general, the formulation of this disclosure is not limited to a particular subject or group of subjects, although human administration will be most common.

The following detailed description is merely exemplary in nature and is not intended to limit the invention or the application and uses of the invention. Furthermore, there is no intention to be bound by any theory presented in the preceding background or the following detailed description.

A multilayer formulation, a method of manufacturing the multilayer formulation, and a method of delivering active agents to a subject are provided herein. The multilayer formulation is described immediately below, followed by a description of the manufacturing method, and the delivery method. It is to be appreciated that the multilayer formulation of this disclosure is not limited to a particular method of manufacture, method of utilization, etc.

### Multilayer Formulation

The multilayer formulation is useful for controlled-release and immediate-release of active agents after administration of the multilayer formulation to a subject. The multilayer formulation comprises a first layer and a second layer disposed adjacent the first layer. The layers may be spaced from one another or in contact. In various embodiments, the layers are abutting or in direct contact on at least one shared surface. In other embodiments, the layers are separated by at least one intervening or spacer layer.

In various embodiments, the multilayer formulation is in the form of a tablet. In further embodiments, the tablet is a bilayer tablet. In certain embodiments, the first and second layers each comprise abutting substantially planar layers which form the tablet. In other embodiments, the multilayer formulation includes three, four, or more layers. One of skill in the art appreciates that many tablet configurations, constructs, shapes, and sizes are possible.

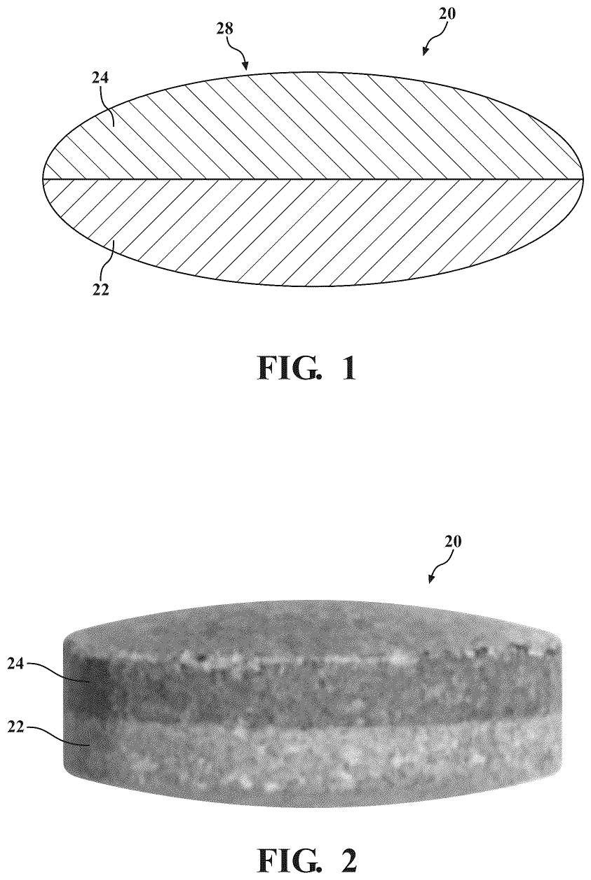

As understood in the art, bilayer tablets are solid dosage forms comprised of two distinct layers within a single tablet unit. Representative bilayer tablets are shown generally in FIGS. 1, 2, and 3A. In these embodiments, the tablet is physically designed in a manner where all surfaces (except one) of the layers are physically not in contact with each other. Applications of such bilayer tablets generally relate to appearance (of two distinct layers), separation of ingredients to avoid physical and/or chemical incompatibility, dual release of active ingredients, etc. In general, the bilayer tablet of this disclosure is especially useful for dual release of the active agents.

Separation of active agents, by locating in them different layers, is beneficial in a number of ways. For example, separation is useful in instances where the active agents are not chemically and/or physically compatible. In addition, separation is useful for providing immediate and extended release of the actives. Further, in instances where the layers include the same or substantially the same actives, a burst effect can first be provided via the immediate release layer, followed by extended release via the extended release layer. The burst effect can be helpful for targeted delivery of the active(s) at one or more locations in the GI tract. Both immediate and extended release of one or more actives is also helpful for distributing them at various locations in the GI tract. This is useful to modify delivery at specific absorption sites, transmit time, interaction and/or co-absorption, of the actives. It is believed that the multilayer formulation ensures optimal accessibility of the actives at specific absorption sites throughout the GI tract. This can lead to better adsorption and use/availability of actives. Another way to interpret this is release of actives in a gradual or staggered manner as the formulation travels in the GI tract after ingestion. This can provide extended or lengthy hours of nutritional support.

Optionally, an outer coating covers the first and second layers. Coverage of the layers may be full or partial. For example, the outer coating may completely envelope or encapsulate the layers. The outer coating may be a conventional coating, but should not be one that negatively impacts desired release profiles of the active agents. This and other aspects of the formulation are detailed further below.

Referring now to the Figures, wherein like numerals indicate like parts throughout the several views, a multilayer formulation is shown generally at 20. In certain embodiments, such as those generally shown in FIGS. 1, 2, 3A, and 3B, the multilayer formulation 20 is a bilayer tablet 20. The bilayer tablet 20 includes a first layer 22, and a second layer 24 adjacent the first layer 22. The first and second layers 22, 24 are different from one another, e.g. the first layer 22 is formed from the controlled-release formulation, and the second layer 24 is formed from the immediate-release formulation. Such formulations are as described herein. The layers 22, 24 can be different in alternate or further ways as well, e.g. they contain different actives, have different amounts of actives, etc. The first and second layers 22, 24 are as described herein. The first and second layers 22, 24 may be of substantially the same or different average thickness with respect to one another. As used herein, it should be appreciated that the terms “first” and “second” are not meant to convey a particular order or importance, but are merely used for ease of reference and description.

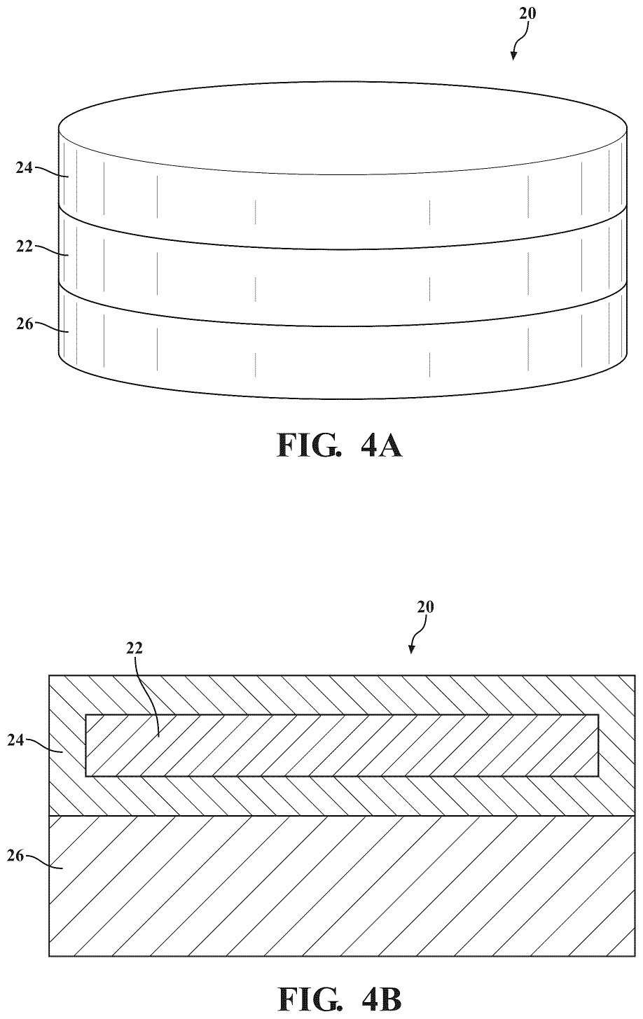

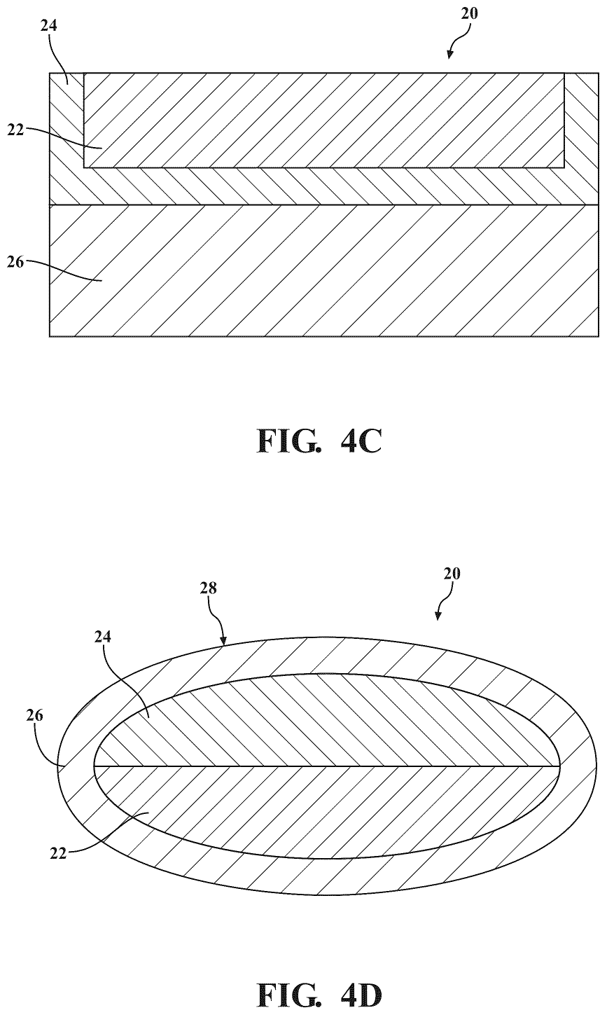

In other embodiments, such as those generally shown in FIGS. 4A, 4B, 4C, and 4D, the multilayer formulation 20 is a trilayer tablet 20, which further includes a third layer 26. The third layer 26 may be the same as or different from one of the first and second layers 22, 24. The third layer 26 may be the same as the first layer 22, the same as the second layer 24, or different from both of the first and second layers 22, 24. For example, the third layer 26 may have a different release rate, contain different actives, have different amounts of actives, etc. The first, second, and third layers 22, 24, 26 may be of substantially the same or different average thickness with respect to one another.

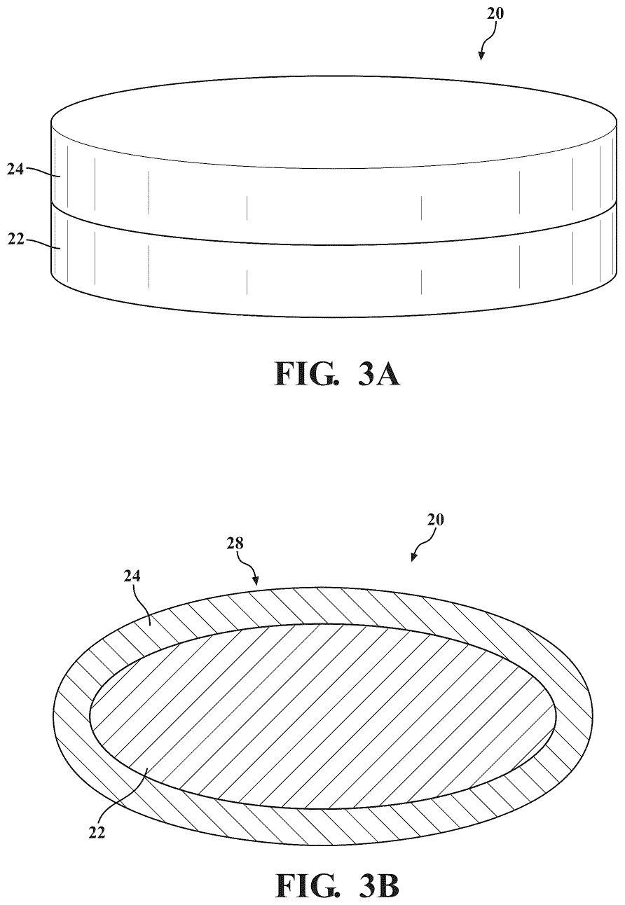

As shown in FIGS. 1, 2, and 3A, the first and second layers 22, 24 are in direct contact with one another. In these embodiments, the layers 22, 24 are generally only in contact on one side or surface. In other embodiments, the first layer 22 is in contact with the second layer 24 on two or more sides or surfaces. For example, as shown in FIG. 3B, the first layer 22 is surrounded or encapsulated by the second layer 24. In other embodiments (not shown), the second layer 24 is surrounded or encapsulated by the first layer 22. In such embodiments, the encapsulation may be full or partial.

In certain embodiments, the layers 22, 24, 26 are sandwiched. For example, as shown in FIG. 4A, the first layer 22 is sandwiched between the second and third layers 24, 26. In this embodiment, the second and third layers 24, 26 are spaced from one another so they are not in contact; whereas the first layer 24 is in direct contact with one side or surface of each of the second and third layers 24, 26. In other embodiments (not shown), the second layer 24 is sandwiched between the first and third layers 22, 26, or the third layer 26 is sandwiched between the first and second layers 22, 24.

Referring to FIG. 4B, the first layer 22 is surrounded or encapsulated by the second layer 24. The third layer 26 is in direct contact with the second layer 24. In other embodiments (not shown), the second layer 24 or third layer 26 is surrounded or encapsulated by the first layer 24. In yet other embodiments (not shown), the first layer 22 or second layer 24 is surrounded or encapsulated by the third layer 26.

Referring to FIG. 4C, the first layer 22 is partially surrounded or encapsulated by the second layer 24. The third layer 26 is in direct contact with the second layer 24. In other embodiments (not shown), the second layer 24 or third layer 26 is partially surrounded or encapsulated by the first layer 24. In yet other embodiments (not shown), the first layer 22 or second layer 24 is partially surrounded or encapsulated by the third layer 26.

Referring to FIG. 4D, the tablet 20 can be a hybrid of the aforementioned embodiments. Specifically, a tablet 20 like that of FIG. 1 can be surrounded or encapsulated by the third layer 26. In other embodiments (not shown), the tablet 20 can have a core comprising different combinations of layers and/or a different outer layer.

In some embodiments, the multilayer formulation 20 comprises a film (or outer) coating 28. Such film coating 28 is optional, but can be useful for imparting various properties as understood in the art. For example, the film coating 28 can be used to improve product appearance, organoleptic properties, shelf life, and/or facilitate swallowing. In certain embodiments, the film coating 28 is an enteric coating; whereas, in other embodiments the film coating 28 would not be classified as an enteric coating. The film coating can be formed from various materials understood in the art, such as a conventional polymer coating. Referring to FIGS. 1, 3B, and 4D, the film coating 28 is disposed on the outer surface of the tablet 20.

### First Layer—Controlled-Release

Referring back to the multilayer formulation in general, the first layer comprises (and/or is formed from) a controlled-release composition. The controlled-release composition comprises a first active agent and a controlled-release excipient. The first active agent may be a conventional active agent, and examples thereof are described further below.

The controlled-release excipient comprises a modified tapioca starch. By “modified”, it is generally meant that the native (or “nonmodified”) tapioca starch is physically and/or chemically modified to change its physical and/or chemical properties. The controlled-release excipient may also be referred to as a controlled-release agent.

In certain embodiments, the controlled-release excipient is (or consists of) the modified tapioca starch. In these or other embodiments, the modified tapioca starch may be the only (release) excipient present in the first layer. Said another way, the first layer can exclude other conventional excipients that are associated with altering release properties of tablet formulations. It is believed that the modified tapioca starch provides for controlled-release of the first active agent. For example, upon administration to a subject, the modified tapioca starch contributes to controlled-release of the first active agent from the first layer of the multilayer formulation.

In certain embodiments, the modified tapioca starch is selected from the group consisting of hydroxypropyl tapioca starch, carboxymethyl tapioca starch, acid-modified tapioca starch, crosslinked modified tapioca starch (e.g., crosslinked carboxymethylguar and modified tapioca starch, crosslinked tapioca starch phosphate, and crosslinked tapioca starch-borate-urea), grafted tapioca starch (e.g., methyl methacrylate grafted hydroxypropyl tapioca starch and ethyl methacrylate grafted tapioca starch), enzyme-catalyzed tapioca starch, pregelatinized tapioca starch, and combinations thereof. Non-limiting examples of suitable modified tapioca starches are commercially available from Ingredion Incorporated of Westchester, IL under the tradename ULTRA-TEX®, such as ULTRA-TEX® 2, ULTRA-TEX® 3, ULTRA-TEX® 4, ULTRA-TEX® 8, ULTRA-TEX® SR, and ULTRA-TEX® 2131.

In various embodiments, the modified tapioca starch comprises or consists of a hydroxypropyl tapioca starch. Suitable hydroxypropyl tapioca starches may be bought commercially or prepared, e.g., by using propylene oxide under alkali condition where hydroxypropyl substitutes are added at the OH positions of anhydroglucose unit (AGU) of native tapioca starch by an ether linkage. See, e.g., the reaction scheme below:

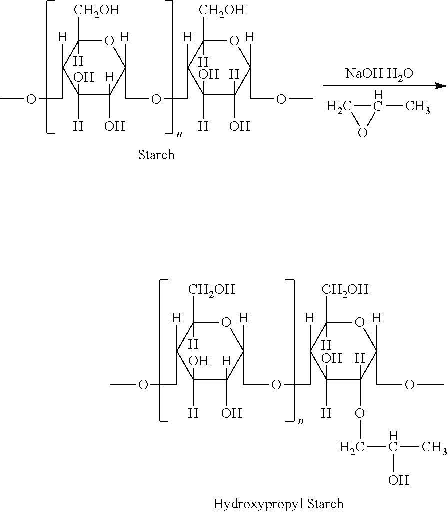

It is to be appreciated that the controlled-release composition may include two or more of the modified tapioca starches listed above. The two or more modified tapioca starches may provide a formulator the ability to control various properties of the controlled-release composition including, but not limited to, formation characteristics, dissolution profile, disintegration profile, and active agent release profiles. The two or more modified tapioca starches may be combined in the form of a heterogeneous blend extending throughout the controlled-release composition, a gradient extending from an interior to an exterior of the controlled-release composition, or discrete layers.

The modified tapioca starch may have or exhibit a variety of properties including, but not limited to, moisture content, pH, viscosity, and density. The modified tapioca starch may have a moisture content of no greater than 15%. The modified tapioca starch may have a pH of from about 4 to about 8 as determined by a 10% w/w slurry. The modified tapioca starch may have a viscosity of from about 500 to about 1200 MVU as determined in accordance with CML-M404H for 15 minutes. In certain embodiments, viscosity of the modified tapioca starch has an effect on formation of the gel from the hydrophilic matrix of the tablet.

In various embodiments, the controlled-release composition is substantially free or completely free of excipients other than the modified tapioca starch. As used herein, the term “substantially free”, generally means that the composition has less than 5 wt. %, optionally less than 2 wt. %, optionally less than 1 wt. %, or optionally approaching 0 wt. %. Typically, the modified tapioca starch is near equivalent, equivalent, or superior to conventional excipients (e.g., HPMC or HAS) in cost, affecting controlled-release of the active agent(s), and marketing potential (e.g., the use of a more “natural” sounding excipient has advantages over more “chemical” sounding excipients in capturing market share by being more “label friendly”).

Many conventional polymeric or “unnatural” excipients are prone to causing confusion or aversion when consumers refer to product labels listing such components by their chemical names. On the other hand, tapioca and thus tapioca starch is well known to many consumers, and is generally considered to be safe for consumption with no adverse effects.

In various embodiments, the controlled-release composition (and thus the first layer) is substantially or completely free of polymeric crosslinking, e.g. such as that imparted by conventional polymeric excipients or the like. In further or other embodiments, the controlled-release composition is substantially or completely free of hydroxypropyl methylcellulose starch (HPMC). In yet further or other embodiments, the controlled-release composition is substantially or completely free of high-amylose starch (HAS). In yet further or other embodiments, the controlled-release composition is substantially or completely free of methylcellulose (MC). In yet further or other embodiments, the controlled-release composition is substantially or completely free of hydroxypropyl cellulose (HPC). In yet further or other embodiments, the controlled-release composition is substantially or completely free of ethyl cellulose (EC).

The first active agent and controlled-release excipient may be present in the controlled-release composition (and thus the first layer) in various weight ratios. That said, it is believed that certain weight ratios impart the first layer with excellent or desirable release properties, which may depend on the particular active agent, amount thereof, size and shape of tablet, etc. Suitable weight ratios may be determined via routine experimentation, and may depend on want or need. In various embodiments, the weight ratio of the first active agent and controlled-release excipient is from about 0.001:1 to about 4:1, optionally from about 0.01:1 to about 4:1, optionally from about 0.1:1 to about 4:1, optionally from about 0.3:1 to about 3.7:1, optionally from about 0.4:1 to about 3.5:1, optionally from about 0.5:1 to about 3.5:1, optionally from about 1:1 to about 3:1, optionally from about 2:1 to about 3:1, or optionally about 2.5:1. Without being limited by any particular theory, it is believed that certain weight ratios are useful for selective active(s) delivery at specific absorption sites throughout the GI tract. Suitable ratios of the active(s) and excipient(s) can be determined via routine experimentation.

The first active agent may be present in the controlled-release composition (and thus the first layer) in any amount. In various embodiments, the first active agent is present in an amount of from about 1 to about 50, optionally about 15 to about 45, optionally about 30 to about 40, or optionally about 35, wt. %, based on the total weight of the controlled-release composition. The first active agent may include one active or a combination of two or more different actives. The remainder of the total weight can be the controlled-release excipient and optionally one or more additives.

The controlled-release excipient may be present in the controlled-release composition (and thus the first layer) in any amount. In various embodiments, controlled-release excipient is present in an amount of from about 1 to about 50, optionally about 5 to about 30, optionally about 10 to about 20, or optionally about 15, wt. %, based on the total weight of the controlled-release composition. The remainder of the total weight can be the first active agent and optionally one or more additives.

The first layer may be of different sizes. In various embodiments, the first layer has a total weight of from about 1 to about 5000, optionally from about 5 to about 3000, optionally from about 50 to about 1500, optionally from about 100 to about 1000, optionally from about 250 to about 1000, or optionally from about 500 to about 1000, mg. The total weight includes the first active agent and controlled-release excipient, e.g. in the ranges described above, and optionally with the remainder being other components as described herein, e.g. in the Examples.

The first active agent and controlled-release excipient may be substantially homogeneously disposed throughout the first layer. The controlled-release excipient being substantially homogeneously disposed throughout the first layer may be referred to as a hydrophilic matrix.

The controlled-release composition is adapted to release the first active agent in a modified way. The first active agent can be released in different amounts over a period of time, with certain release thresholds or targets at 1, 3, and 6 hours after ingestion, for example. In certain embodiments, a majority to all of the first active agent is released from the first layer after about 6 to about 12, optionally about 6 to about 10, or optionally about 6 to about 8, hours, after ingestion of the multilayer formulation.

In various embodiments, no greater than 50 wt. % of the first active agent is released within 1 hour of ingestion of the multilayer formulation by a subject, based on a total weight of the first active agent present in the controlled-release composition. In certain embodiments, the controlled-release composition is adapted to release no greater than 45 wt. %, or optionally no greater than 40 wt. %, of the first active agent within 1 hour of ingestion of the multilayer formulation by the subject.

In further or other embodiments, not less than 30 wt. % of the first active agent is released within 3 hours of ingestion of the multilayer formulation by a subject, based on a total weight of the first active agent present in the controlled-release composition. In certain embodiments, the controlled-release composition is adapted to release not less than 35 wt. %, or optionally not less than 40 wt. %, of the first active agent within 3 hours of ingestion of the multilayer formulation by the subject.

In yet further or other embodiments, not less than 55 wt. % of the first active agent is released within 6 hours after ingestion of the multilayer formulation by the subject, based on a total weight of the first active agent present in the controlled-release composition. In certain embodiments, the controlled-release composition is adapted to release not less than 60 wt. %, or optionally not less than 65 wt. %, of the first active agent within 6 hours after ingestion of the multilayer formulation by the subject. In these or other embodiments, the released amounts described above are based on the total weight (or total amount) of the first active agent present in the multilayer formulation as a whole.

In various embodiments, the first layer is adapted to inhibit complete disintegration for at least 10, optionally for at least 30, or optionally for at least 40, minutes, after ingestion by a subject. Thus, the first layer may be defined as delayed-release prior to complete disintegration and extended-release after complete disintegration. As utilized herein, the phrase “complete disintegration” is generally defined as that state in which any residue of the first layer, except fragments of any insoluble coating or capsule shell, if used, remaining on a screen of a test apparatus or adhering to a lower surface of a disk, if used, is a soft mass having no palpably firm core, in accordance with the method described in the Revised Bulletin dated Mar. 1, 2010 entitled “<2040> Disintegration and Dissolution of Dietary Supplements” by The United States Pharmacopeial Convention.

In various embodiments, during disintegration, and after complete disintegration, of the first layer, the fluids within the stomach cavity may react with the controlled-release excipient (or the hydrophilic matrix) of the first layer to form a gel. The gel may inhibit release of the first active agent for a pre-determined amount of time after entering the stomach cavity of a subject.

### Second Layer—Immediate-Release

The second layer comprises (and/or is formed from) an immediate-release composition. The an immediate-release composition is different from the controlled-release composition of the first layer. In certain embodiments, the immediate-release composition is a conventional composition understood in the art. Examples of such, as well as other conventional components which may be used in embodiments of this disclosure, are described in U.S. Pub. No. 2017/0281666, the disclosure of which is incorporated herein by reference in its entirety.

The immediate-release composition comprises a second active agent and an excipient. The second active agent may be the same as or different from the first active agent. In various embodiments, the active agents are different, e.g. in type and/or amount. In certain embodiments, the first and second active agents are the same or are substantially the same. This allows for an initial burst effect followed by a sustained slow effect with respect to the active agent(s). The second active agent may be a conventional active agent, and examples thereof are described in U.S. Pub. No. 2017/0281666 and further below.

The excipient of the immediate-release composition is different from the controlled-release excipient of the controlled-release composition. The excipient may be a conventional excipient, and examples thereof are described in U.S. Pub. No. 2017/0281666, and further below as “Other Components” and in the Examples.

It is to be appreciated that the excipient may be a single component or a blend of components, and such components may also be referred to in the art by other names, such as binders, diluents, bulking agents, or the like. This is because unlike the controlled-release excipient, the excipient of the immediate-release composition generally does not modify or control release of the second active agent, but merely acts as a temporary vehicle or carrier until the multilayer composition is administered, e.g. swallowed by a subject.

In various embodiments, the immediate-release composition (and thus the second layer) is substantially or completely free of polymeric crosslinking, e.g. such as that imparted by conventional polymeric excipients or the like. In further or other embodiments, the immediate-release composition is substantially or completely free of HPMC. In yet further or other embodiments, the immediate-release composition is substantially or completely free of HAS. In yet further or other embodiments, the immediate-release composition is substantially or completely free of MC. In yet further or other embodiments, the immediate-release composition is substantially or completely free of HPC. In yet further or other embodiments, the immediate-release composition is substantially or completely free of EC. Similar to description above for the first layer, exclusion of certain components from the second layer can make the overall multilayer component more label friendly to patients, consumers, etc. For example, it can beneficial to have components that are that less “chemical sounding.” There can also be cost advantages for certain, more natural components relative to their synthetic or less natural counterparts.

The second active agent and excipient may be present in the immediate-release composition (and thus the second layer) in various weight ratios. That said, it is believed that certain ratios may impart the second layer with excellent or desirable release properties, which may depend on the particular active agent, amount thereof, size and shape of tablet, etc. Suitable ratios may be determined via routine experimentation, and may depend on want or need.

The second active agent may be present in the immediate-release composition (and thus the second layer) in any amount. In various embodiments, the second active agent is present in an amount of from about 1 to about 50, optionally about 15 to about 45, optionally about 30 to about 40, or optionally about 35, wt. %, based on the total weight of the immediate-release composition. The second active agent may include one active or a combination of two or more different actives. The remainder of the total weight can be the excipient and optionally one or more additives.

The excipient may be present in the immediate-release composition (and thus the second layer) in any amount. The excipient may include one excipient or a combination of two or more different excipients. Suitable amounts may be determined via routine experimentation, and may depend on want or need.

The second layer may be of different sizes. In various embodiments, the second layer has a total weight of from about 1 to about 5000, optionally from about 5 to about 3000, optionally from about 50 to about 1500, optionally from about 100 to about 1000, optionally from about 250 to about 1000, or optionally from about 500 to about 1000, mg. The total weight includes the second active agent and excipient, e.g. in the ranges described above, and optionally with the remainder being other components as described herein, e.g. in the Examples.

The excipient may be present in the immediate-release composition (and thus the second layer) in any amount. In various embodiments, excipient is present in an amount of from about 1 to about 50, optionally about 5 to about 30, optionally about 10 to about 20, or optionally about 15, wt. %, based on the total weight of the immediate-release composition. The remainder of the total weight can be the second active agent and optionally one or more additives.

The second active agent and excipient may be substantially homogeneously disposed throughout the second layer.

The immediate-release composition is adapted to release the second active agent in a manner different from that of first active agent release. The second active agent can be released in different amounts over a period of time, with certain release thresholds or targets at 1 hour after ingestion, for example. In certain embodiments, a majority to all of the second active agent is released from the second layer after about 30 minutes to about 2.5 hours, optionally after about 1 to about 2 hour(s), or optionally about 1, hour(s) after ingestion of the multilayer formulation.

In various embodiments, not less than 60 wt. % of the second active agent is released within 1 hour after ingestion of the multilayer formulation by a subject, based on a total weight of the second active agent present in the immediate-release composition. In certain embodiments, the immediate-release composition is adapted to release not less than 65 wt. %, optionally not less than 70 wt. %, or optionally not less than 75 wt. %, of the second active agent within 1 hour after ingestion of the multilayer formulation by the subject, based on a total weight of the second active agent present in the immediate-release composition. In these or other embodiments, the released amounts described above are based on the total weight (or total amount) of the second active agent present in the multilayer formulation as a whole.

### Active Agents

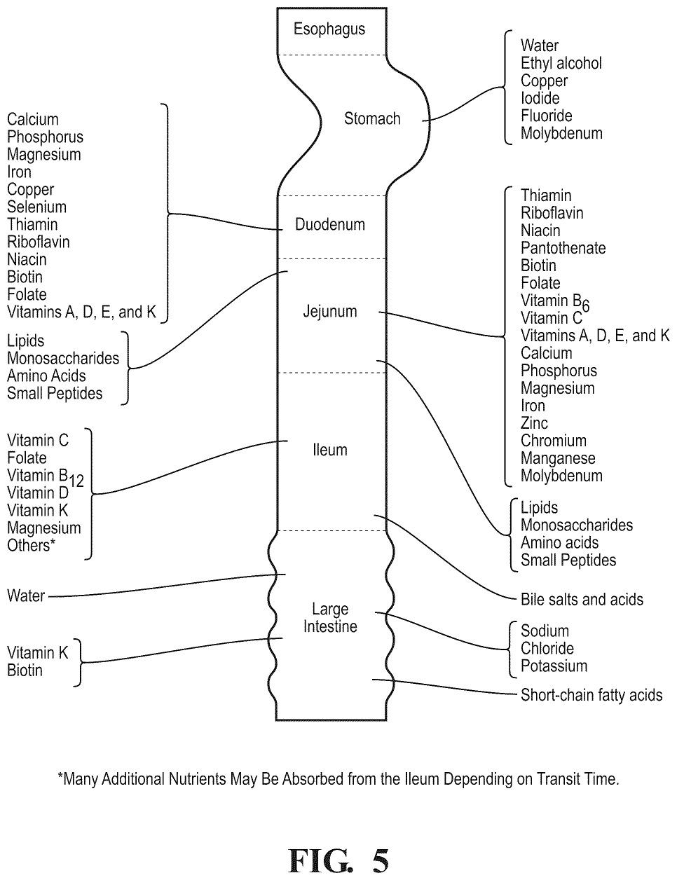

The multilayer formulation is not limited to particular active agents. For example, suitable actives may be selected based on desire or need. In various embodiments, each of the active agents is individually selected from the group consisting of vitamins, minerals, botanicals, phytochemicals, and combinations thereof. Phytochemicals may be obtained or separated from botanicals, remain within botanicals, or be separate components from botanicals. As understood in the art, botanicals may be plant material, or based on plant material, such as extracts. Various extraction methods are possible, and various parts of plants may be used. Botanicals are commercially available from numerous suppliers. In certain embodiments, the active agent(s) for at least one or for each layer can be selected based on the presence of preferential absorption sites throughout the GI tract (see, e.g., FIG. 5.).

In various embodiments, each of the active agents is individually, or optionally at least the first active agent is, selected from the group consisting of zinc, chromium, manganese, iron, magnesium, calcium, vitamin C, vitamin B5, vitamin B6, thiamine, niacin, vitamin B12, and combinations thereof. In further or other embodiments, each of the active agents is individually, or optionally the second active agent is, selected from the group consisting of copper, iodine, molybdenum, calcium, phosphorous, boron, selenium, vitamin A, vitamin E, vitamin D, vitamin K, vitamin B12, biotin, folic acid, riboflavin, and combinations thereof. It is to be appreciated that each of the active agents may include one or more different active compounds or components, e.g. two vitamins, a vitamin and a mineral, etc.

Further examples of suitable vitamins include, but are not limited to, vitamin A, vitamin B1, vitamin B2, vitamin B3, vitamin B5, vitamin B6, folic acid (vitamin B9), vitamin B12, vitamin B complex, ascorbic acid (vitamin C), vitamin D, vitamin D2, vitamin D3, vitamin E, vitamin G, vitamin H, vitamin K, vitamin M, vitamin O, and vitamin Q10. In various embodiments, at least one of the active agents comprises ascorbic acid. In certain embodiments, the weight ratio of the ascorbic acid and controlled-release excipient is from about 1.5:1 to about 3.5:1, optionally from about 2:1 to about 3.5:1, or optionally about 3:1. In further or other embodiments, at least one of the active agents comprises folic acid. In certain embodiments, the weight ratio of the folic acid and controlled-release excipient is from about 0.4:1 to about 3:1, optionally from about 1:1 to about 2.5:1, or optionally about 2:1.

Further examples of suitable minerals include, but are not limited to, iron, iodine, magnesium, zinc, selenium, copper, calcium, manganese, silicon, molybdenum, vanadium, boron, nickel, tin phosphorus, chromium, cobalt, chloride, sodium, sulfur, potassium, and trace minerals. In various embodiments, at least one of the active agents comprises a mineral, such as iron. In certain embodiments, the weight ratio of the mineral and controlled-release excipient is from about 0.1:1 to about 4:1, optionally from about 0.3:1 to about 3.7:1, or optionally from about 0.4:1 to about 3.5:1.

Further examples of suitable phytochemicals include, but are not limited to, phytoestrogens (e.g., isoflavones), lignans, catechins, phenolic acids, saponins, flavonoids, alkaloids (e.g. caffeine), and other non-vitamin chemicals derived from plants. In various embodiments, at least one of the active agents comprises caffeine. In certain embodiments, the weight ratio of the caffeine and controlled-release excipient is from about 0.4:1 to about 3:1, optionally from about 1:1 to about 2.5:1, or optionally from about 1.5:1 to about 2:1.

In various embodiments, at least one of the compositions (and thus corresponding layer(s)) can include or comprise other compounds including, but not limited to, 5-HTP (5-hydroxytryptophan), 7-keto-DHEA (dehydroepiandrosterone), acetate, acetyl-L-carnitine, AE-941, α-carotene, α-hydroxy acids, α-aminohydrocinnamic acid, α-ketoglutarate, α-galactosidase, α-linolenic acid, α-lipoic acid, α-tocopherol, androstenediol, androstenedione, arginine, aspartic acid (aspartate), β-alanine, β-alanyl-L-histidine, β-carotene, β-cryptoxanthin, β-D-fructofuranosidase, betadine, β-glucan, β-glycans, betaine, β-sitosterol, β-tocopherol, calcium carbonate matrix, calcium phosphate, caprylic acid, canthaxanthin, CDP-choline, chelated calcium, cholecalciferol, choline, chondroitin sulfate, citicoline, citric acid, creatine, cryptoxanthin, cysteine, D-calcium pantothenate, dehydroepiandrosterone, delta-tocopherol, dexpanthenol, dextran-iron, DGL (deglycyrrhiziated licorice), EA (Dehydroepiandrosterone), dibencozide, dichloroacetate, dimethylglycine, dimethylsulfone, disodium disuccinate astaxanthin, D,L-phenylalanine, DMAE (Dimethylaminoethanol), D-mannose, DMSO (dimethyl sulfoxide), docosahexaenoic acid, docusate sodium, eburnamenine-14-carboxylic acid, EDTA (ethylenediamine tetraacetic acid), EFA (essential fatty acid), ellagic acid, eicosapentaenoic acid, ferrous gluconate, ferrous sulfate, 5-hydroxytryptophan, flavonoid, folacin, folate, forskolin, fructo-oligosaccharides, GABA (gamma-aminobutyric acid), galanthamine hydrobromide, γ-carotene, γ-linolenic acid, γ-oryzanol, γ-glutamylcysteinylglycine, γ-tocopherol, glucosamine, glucosamine sulfate, glutamine, glutamic acid, glutathione, glycerol, glycerophosphocholine, glycine, histidine, HMB (β-hydroxy-β-methylbutyrate monohydrate), hydroxocobalamin, hydroxycitric acid, hydroxymethylbutyrate, hydroxytryptophan, hyoscine butylbromide (scopolamine), hydroxylysine, hydroxyproline, hypoxanthine riboside, indole-3-carbinol, inosine, inositol hexanicotinate, inositol hexaphosphate, isoascorbic acid, isoflavones, isoleucine, lactic acid, L-arginine, L-ascorbic acid, L-asparagine, L-carnitine, L-Dopa, leucine, L-phenylalanine, L-tryptophan, luzindole, lycopene, lysine, malic acid, mesoglycan, methionine, methylcobalamin, methylguanidine acetic acid, methylsulfonylmethane, monounsaturated fatty acid, N-3 fatty acids, N-acetyl cysteine, N-acetyl D-glucosamine, N-acetyl-5-methoxytryptamine, N-acetylaspartic acid, NADH, niacin, nicotinamide adenine dinucleotide, nordihydroguaiaretic acid (NDGA), octacosanol, octanoic acid, oleuropein, omega-3 fatty acids, omega-6 fatty acids, omega-9 fatty acid, PABA (para-aminobenzoic acid), pangamic acid, pantethine, pantothenic acid, pantothenol, perillyl alcohol, PGG-glucan, phenylacetate, phosphatidylcholine, phosphatidylserine, phytoestrogen, phytonadione, phytosterols, polyphenols, polysaccharide-K, polyunsaturated fatty acids, polyvinylpyrrolidone-iodine, potassium, potassium aspartate, potassium phosphate, povidone-iodine, pregnenolone, progesterone, provitamin a, pteroylglutamic acid, pyridoxine, pyridoxal-5-phosphate, quercetin, quercetin-3-rhamnoglucoside, quercetin-3-rutinoside, quinine, resveratrol, retinol, riboflavin, riboflavin-5-phosphate, salicin, salicylate, SAM-e (S-adenosylmethionine), sitostanol, sitosterol, sitosterolins, sodium alginate, sodium ascorbate, sodium chloride, sodium ferric gluconate, sodium iodide, sodium phenylacetate, sodium phosphate, sorbic acid, stigmasterol, sulforaphane, synephrine, tannic acid, theanine, theobromine, thiamin, thioctic acid, tocopherols, tocotrienols, triacylglycerol lipase, tricholine citrate (TRI), troxerutin, tryptophan, tyrosine, acetyl-L-tyrosine, ubidecarenone, ubiquinone, urosolic acid, usnic acid, valine, xylitol, or zeaxanthin.

In specific embodiments, the first and/or second active agent(s) can include or comprise: one or more minerals, such as zinc (e.g. 10 mg), chromium (e.g. 50 mcg), manganese (e.g. 4 mg), iron (e.g. 21 mg), or combinations thereof; and one or more vitamins, such as vitamin C (e.g. 40 mg), vitamin B5 (e.g. 5 mg), vitamin B6 (e.g. 2 mg), thiamine (e.g. 1 mg), niacin (e.g. 12 mcg), or combinations thereof. In addition, the first and/or second active agent(s) can include or comprise one or more minerals, such as copper (e.g. 1.7 mg), iodine (e.g. 150 mcg), molybdenum (e.g. 45 mcg), selenium (e.g. 40 mcg), or combinations thereof; vitamins, such as vitamin A (e.g. 600 mcg), vitamin E (e.g. 10 mg), vitamin D (e.g. 400 IU), vitamin K (e.g. 55 mcg), vitamin B12 (e.g. 1 mcg), biotin (e.g. 30 mcg), dietary folate (e.g. 200 mcg), riboflavin (e.g. 1.1 mg), or combinations thereof. Moreover, the first and/or second active agent(s) can include or comprise one or more botanicals, such as acerola powder, purple carrot juice powder, elderberry extract, Centella asiatica (e.g. 25:1) extract, Acorus calamus (e.g. 12:1) extract, Boerhavia diffusa (e.g. 25:1) extract, Syzygium aromaticum (e.g. 20:1) extract, or combinations thereof. Centella asiatica may also be referred to as gotu kola. Various other amounts and combinations of actives (e.g., vitamins, minerals, and botanicals) are also contemplated. In various embodiments, the multilayer formulation has 13 vitamins and 11 minerals at quantities as per the RDA guidelines laid down by ICMR, NIN adopted by FSSAI.

### Other Components

In addition to the active agents and excipients, each of the compositions may individually include one or more components or additives so long as the desired release profiles of the active agents are not impaired. The one or more additives may include, but are not limited to, a diluent, a filler, a flavoring agent (e.g. an acidulant, a sweetener, a perfume, etc.), a lubricant, a binder, a colorant, a surfactant, a stabilizing agent, a blowing agent, or combinations thereof. As introduced above, excipients can be further classified as other components. Specifically, excipients used in oral solid dosage forms have been classified based on their functionality into groups such as diluents, disintegrants, binders, compression aids, granulating agents, glidants, lubricants, release-controlling polymers, stabilizers (such as antioxidants, chelators, and pH-modifiers), film-coating polymers, coating agents, vehicles, plasticizers, surfactants, colorants, sweeteners, and flavors.

In various embodiments, at least one of the compositions comprises at least one component selected from the group consisting of binders, lubricants, glidants, and combinations thereof. In certain embodiments, the immediate-release composition comprises at least one disintegrant. In general, such additional components can be those known in the art. For example, U.S. Pub. No. 2017/0281666 describes a number of conventional components suitable for formulating bilayer tablets, suitable amounts of such components, and methods of forming tablets therefrom.

In various embodiments, at least one of the compositions includes one or more compounds including, but not limited to, methylcellulose, hydroxypropyl methylcellulose, ethyl cellulose, cellulose acetate phthalate, acacia, gums, wax, glycerol monostearate, acrylic acid polymers and copolymers, methacrylic acid, methyl acrylate, ethyl acrylate, methyl methacrylate, ethyl methacrylate, lactose, calcium sulfate, calcium phosphate dibasic, sugar, microcrystalline cellulose (MCC), starch, sodium starch glycolate, polyvinylpyrrolidone, polyethylene glycol, and magnesium stearate. Combinations of such components can be utilized, and such components and other components used in conventional tablets are understood in the art.

In certain embodiments, the modified tapioca starch is used as a sole natural excipient in the multilayer formulations. In such embodiments, no polymers are used as release-controlling excipients or additives.

As used herein, “diluents” may be inert substances added to increase the bulk of at least one the compositions to make the tablet a practical size for compression. As such, they may also be referred to as bulking agents. Commonly used diluents include, but are not limited to, microcrystalline cellulose (MCC), wood cellulose, corn starch, modified corn starch, (tri) calcium phosphate, calcium sulfate, lactose, kaolin, mannitol, sodium chloride, dry starch, (powdered) sugar, dextrose, mannitol, sorbitol, and the like. The diluent/bulking agent may be used alone or in various mixtures, and utilized in any amount known in the art for oral compositions.

As used herein, “flavoring agents” are compounds designed to give the composition a more palatable taste. Flavoring agents vary considerably in their chemical structure, ranging from simple esters, alcohols, and aldehydes to carbohydrates and complex volatile oils. Synthetic flavors of almost any desired type are now available and are well known in the art. If hard taste, acid taste or bitter taste derived from starting materials may be suppressed by seasoning or flavoring, the acidulant (e.g., citric acid, tartaric acid, malic acid, ascorbic acid, etc.), the sweetener (e.g. sodium saccharin, dipotassium glycyrrhizinate, aspartame, stevia, thaumatin, etc.), or the perfume (e.g. various fruit perfumes containing lemon oil, orange oil or strawberry, and yoghurt, mint, menthol, etc.) may be included in the composition. The flavoring agent may be used alone or in various mixtures, and utilized in any amount known in the art for oral compositions.

As used herein, “lubricants” are materials that perform a number of functions relating to compositions. In certain embodiments, like tablet manufacture, the lubricants perform one or more functions such as improving the rate of flow of the tablet granulation, preventing adhesion of the tablet material to the surface of dies and punches, reducing interparticle friction, and facilitating the ejection of the tablets from a die cavity. Examples of suitable lubricants include, but are not limited to, zinc stearate, gum arabic powder, cacao butter, carnauba wax, carmellosecalcium, carmellosesodium, caropeptide, aqueous silicon dioxide, dried aluminum hydroxide gel, glycerin, magnesium silicate, light anhydrous silicic acid, light liquid paraffin, crystalline cellulose, hardened oil, synthetic aluminum silicate, sesame oil, flour starch, white beeswax, magnesium oxide, dimethyl polysiloxane, potassium sodium tartrate, sucrose fatty acid ester, glycerin fatty acid ester, silicon resin, aluminum hydroxide gel, stearyl alcohol, stearic acid, aluminum stearate, calcium stearate, polyoxyl stearate, magnesium stearate, cetanol, gelatin, talc, magnesium carbonate, precipitated calcium carbonate, cornstarch, lactose, hard fat, saccharose, potato starch, hydroxypropylcellulose, fumaric acid, sodium stearyl fumarate, polyethylene glycol, polyoxyethylene polyoxypropylene glycol, polysorbate, beeswax, magnesium aluminometasilicate, methylcellulose, Japan wax, glycerin monostearate, sodium lauryl sulfate, calcium sulfate, magnesium sulfate, liquid paraffin, phosphoric acid, palmitic acid, and hydrogenated vegetable oils and fats. The lubricant may be used alone or in various mixtures, and utilized in any amount known in the art for oral compositions.

As used herein, “binders” are agents used to impart cohesive qualities to powdered materials. Binders, or “granulators” as they are sometimes known, impart a cohesiveness to the tablet formulation, which ensures the tablet remaining intact after compression, as well as improving the free-flowing qualities by the formulation of granules of desired hardness and size. Materials commonly used as binders include starch, such as corn starch and pregelatinized starch; gelatin; sugars, such as sucrose, glucose, dextrose, molasses, and lactose; natural and synthetic gums, such as gum acacia, sodium alginate, extract of Irish moss, panwar gum, ghatti gum, mucilage of isapol husks, carboxymethylcellulose, methylcellulose, polyvinylpyrrolidone (PVP), Veegum, microcrystalline cellulose, microcrystalline dextrose, amylose, larch arabogalactan, ethyl cellulose, cellulose acetate, and the like. The binder may be used alone or in various mixtures, and utilized in any amount known in the art for oral compositions.

As used herein, “colorants” are agents that give the composition a more pleasing appearance, and in addition help the manufacturer to control the product during its preparation and help the user to identify the product. Any of the approved certified water-soluble FD&C dyes, mixtures thereof, or their corresponding lakes may be used to color tablets. A color lake is the combination by adsorption of a water-soluble dye to a hydrous oxide of a heavy metal, resulting in an insoluble form of the dye. The colorant may be used alone or in various mixtures, and utilized in any amount known in the art for oral compositions.

Other conventional ingredients that may optionally be present in the multilayer formulation include preservatives, stabilizers, anti-adherents or silica flow conditioners or glidants, such as SYLOID® silicon dioxide. Such ingredients may be used alone or in various mixtures, and utilized in any amount known in the art for oral compositions.

### Method of Manufacture

A method of manufacturing the multilayer formulation is also provided. The method comprises providing the controlled-release composition and providing the immediate-release composition. The method further comprises forming the first layer from the controlled-release composition, and forming the second layer from the immediate-release composition. The method yet further comprises combining the first and second layers to form the multilayer formulation. The steps of forming and combining may be separate or may occur simultaneously.

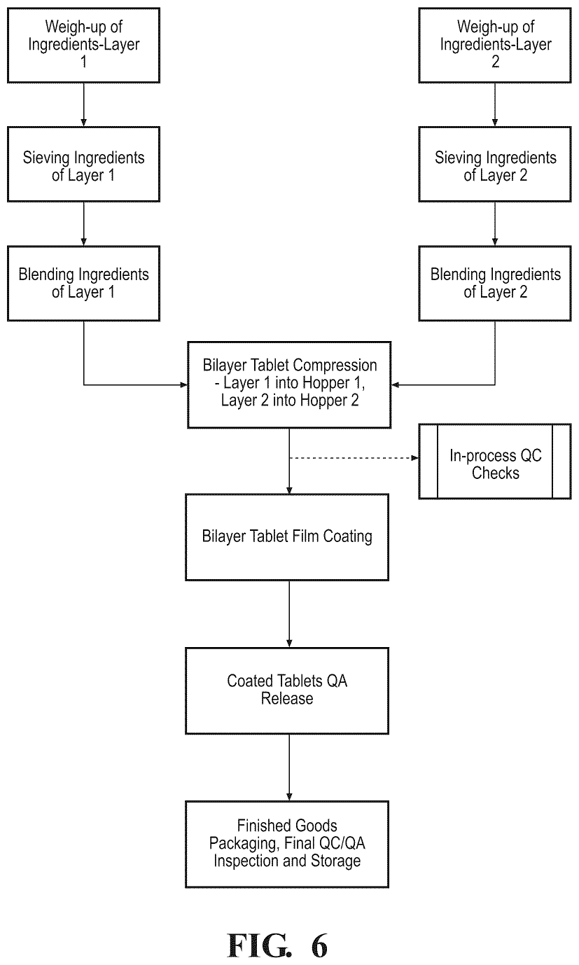

In various embodiments, bilayer tablet blends could be prepared using direct compression or granulation blends depending on the application and physicochemical properties of the ingredients. Production of bilayer tablets is quite similar to a traditional single layer tablet. A difference is in the manufacturing press design, having two hopper and two compression cycles for the two layers of tablets. As one example, a brief manufacturing process is outlined in FIG. 6.

In various embodiments, the multilayer formulation is formed utilizing direct compression or granulation (e.g. wet granulation or dry granulation). In certain embodiments, the multilayer formulation is formed utilizing direct compression. It is to be appreciated that controlled-release of the first active agent from the multilayer formulation can be impacted by the method utilized to form the multilayer formulation. It is to be appreciated that the release profile of the multilayer formulation achieved by a certain method of manufacture may not be consistent with other methods, albeit the ratios of the active agents and excipients being the same.

In certain embodiments, formation of the gel from the hydrophilic matrix of the multilayer formulation, and the resulting release profile, are dependent on the method of manufacture of the tablet. For example, wet granulation formation of the multilayer formulation includes agglomeration of particles using a liquid binder that may affect formation of the gel from the hydrophilic matrix of the multilayer formulation.

The method may further include forming the outer coating on the multilayer formulation. The outer coating may be formed from conventional coating compositions known in the art. The outer coating may also be formed utilizing any method known in the art for forming coatings on tablets.

Conventional coating formulations may include one or more film-formers or binder, such as a hydrophilic polymer like hydroxy-propylmethyl cellulose (HPMC) and a hydrophobic polymer like ethyl cellulose, cellulose acetate, polyvinyl alcohol-maleic anhydride copolymers, acrylic copolymers, β-pinene polymers, glyceryl esters of wood resins and the like, and one or more plasticizers, such as polyethylene glycol, triethyl citrate, diethyl phthalate, propylene glycol, glycerin, butyl phthalate, castor oil, waxes such as carnauba wax, and the like.

The film-formers are generally applied from a solvent system containing one or more solvents including water, alcohols like methyl alcohol, ethyl alcohol or isopropyl alcohol, ketones like acetone, or ethylmethyl ketone, chlorinated hydrocarbons like methylene chloride, dichloroethane, and 1,1,1-trichloroethane. Combinations of film-formers and/or solvents cane be used. In various embodiments, the outer coating is different from a conventional enteric coating, as such enteric coatings may prevent release of the second active agent from the second layer.

The multilayer formulation be of different sizes. In various embodiments, the multilayer formulation has a total weight of from about 2 to about 10000, optionally from about 10 to about 6000, optionally from about 100 to about 3000, optionally from about 200 to about 2000, optionally from about 500 to about 2000, optionally from about 1000 to about 1750, or optionally from about 1250 to about 1500, mg. In general, smaller tablets are preferred for ease of swallowing. Thus, certain dosages may be split into two or more smaller tablets.

### Method of Administering

A method of delivering active agents to a subject is also provided. The method comprises providing the multilayer formulation. The method further comprising administering the multilayer formulation to a subject. For example, the multilayer formulation may be administered orally to the subject. In various embodiments, the multilayer formulation is administered as a bilayer tablet. In many embodiments, the multilayer formulation may be referred to as a nutritional supplemental, a dietary supplement, or the like.

Typically, the multilayer formulation is administered (or ingested) orally, e.g. via the mouth (or “per os”). More typically, at least a portion of the multilayer formulation is administered (or digested) enternally, e.g. via the gastrointestinal (GI) track (or “enteros”). The subject is typically a human, and can include men and women of various ages. The method/formulation of this disclosure is not limited to a particular subject.

The multilayer formulation may be administered as needed, daily, several times per day or in any suitable regimen such that the desired outcome is achieved. Generally, a regimen includes ingestion of the multilayer formulation once or twice daily to include an administration in the morning and/or an administration in the evening. The amount and/or frequency of administration of the multilayer formulation may depend on several factors, including the level of desired results and the specific multilayer formulation.

After administration, a percentage of total second active agent released from the multilayer formulation is generally greater than a percentage of total first active agent released from the multilayer formulation during the same period of time, e.g., within the first hour after administration. In this way, the active agents may be released at different locations in a subject's GI tract. For example, the second active agent is predominantly released “upstream” of the first active agent, although there may also be some overlap in release in the subject's GI tract. A simplified GI tract is illustrated in FIG. 5.

In general, the first layer provides controlled-release of the first active agent at a second, later, or subsequent location in the GI tract of the subject relative to release provided by the second layer. In certain embodiments, the second location in the GI tract of the subject is selected from the group consisting of the duodenum, jejunum, ileum, large intestine, and combinations thereof. The jejunum and ileum may also be referred to as the small intestine. In further embodiments, the targeted absorption sites are the jejunum, ileum, and further GI tract.

The second layer provides immediate-release of the second active agent at a first, prior, or initial location in the GI tract of the subject relative to release provided by the first layer. In certain embodiments, the first location in the GI tract of the subject is selected from the group consisting of the stomach, duodenum, and combinations thereof. In further embodiments, the targeted absorption sites are the stomach and duodenum.

Thus, it should be appreciated that in the GI tract, the second location may be the same as the first location (e.g., there may be some overlap in the lower duodenum), further downstream in the GI tract than the first location, or both. To maximize absorption efficiency of different active agents at different locations in the GI tract, the two layers provide dual action with controlled-release layer generally providing for modified release of the first active agent, and the intermediate-release layer generally providing for instant or near instant release of the second active agent.

One advantage of separating certain active agents into layers is that it allows control of the release from the layers at a time and location that corresponds to the location of optimal absorption in the GI tract. This separation also limits the potential for unwanted binding to food and other particles that may reduce the bioavailability of certain actives. By separating various components in the different layers of the formulation, they can aid each other to optimize absorption, and keep the interference of components in the GI tract minimized.

Another advantage of separating the active agents into layers is that the modified release of certain actives helps to limit the waste associated with large, instant release dosages by minimizing the saturation of the competition for transporters within the small intestine. Certain actives, such as vitamin B12, can also be susceptible to acid degradation in the stomach, so such actives may be better suited for modified- or controlled-release (although immediate-release is also possible).

In certain embodiments, the multilayer formulation is adapted to disintegrate a pre-determined amount of time after entering the stomach of a subject. Fluid within the stomach may interact with the multilayer formulation to support disintegration of the multilayer formulation. In various embodiments, during and after disintegration of the multilayer formulation, the fluids within the stomach may react with one or more of the excipients of the multilayer formulation to form a gel, e.g. the modified tapioca starch. Thus, the gel may inhibit release of the active agents a pre-determined amount of time after entering the stomach of a subject.

### Examples

The following examples are included to demonstrate various embodiments as contemplated herein. It should be appreciated by those of skill in the art that the techniques disclosed in the examples which follow represent techniques discovered by the inventor(s) to function well in the practice of the invention, and thus can be considered to constitute desirable modes for its practice. However, those of skill in the art should, in light of the present disclosure, appreciate that many changes can be made in the specific embodiments which are disclosed and still obtain a like or similar result without departing from the spirit and scope of the invention.

All percentages are in wt. % and all measurements are conducted at ˜37° C. unless indicated otherwise. It is to be appreciated that percent dissolutions of the active agents in Tables II, IV, VI, VIII, IX, X, and XI may have an error of +/−10%. It should also be appreciated that for results over 100%, such results can be attributed to one or more factors, including, for example: 1) tablet weight variation from manufacture (higher or lower than expected weight), which can lead to higher or lower input than target; 2) potency variation of the active raw material assays; and/or 3) analytical error in quantification of the amount(s) dissolved.

### Example I: Evaluation of Compositions Including Ascorbic Acid

Exemplary and comparative compositions including ascorbic acid (Active Agents I and II) were formed as provided in Table I below. The compositions were formed into tablets using direct compression. The tablets included 0.8 to 1.2 grams of composition. The tablets had an ovular configuration. Exemplary compositions (Ex. Form. 1-4) included ascorbic acid and a modified tapioca starch in a weight ratio of from about 0.1:1 to about 4:1. Comparative compositions (Comp. Form. 1-5) included various comparative excipients or included ascorbic acid and the excipient in a weight ratio greater than or less than 0.1:1 to about 4:1.

TABLE I
Compositions including Ascorbic Acid

| | Ex. Form No. (wt. %) | | | | Comp. Form. No. (wt. %) | | | | |
|---|---|---|---|---|---|---|---|---|---|
| Component | 1 | 2 | 3 | 4 | 1 | 2 | 3 | 4 | 5 |
| Active Agent I | 58.45 | 48.71 | 58.45 | 58.45 | 73.06 | 64.94 | 58.45 | 58.45 | 58.45 |
| Active Agent II | 12 | 10 | 12 | 12 | 15 | 13.33 | 12 | 12 | 12 |
| Active Agent III | 3.5 | 2.92 | 3.5 | 3.5 | 4.38 | 3.89 | 3.5 | 3.5 | 3.5 |
| Excipient I | 23.25 | 36.05 | 23.25 | 23.25 | 4.76 | 15.03 | 0 | 0 | 0 |
| Excipient II | 0 | 0 | 0 | 0 | 0 | 0 | 23.25 | 0 | 0 |
| Excipient III | 0 | 0 | 0 | 0 | 0 | 0 | 0 | 23.25 | 0 |
| Excipient IV | 0 | 0 | 0 | 0 | 0 | 0 | 0 | 0 | 23.25 |
| Additive I | 2 | 1.65 | 2 | 2 | 2 | 2 | 2 | 2 | 2 |
| Additive II | 0.8 | 0.67 | 0.8 | 0.8 | 0.8 | 0.8 | 0.8 | 0.8 | 0.8 |
| TOTAL | 100 | 100 | 100 | 100 | 100 | 100 | 100 | 100 | 100 |

Active Agent I is a commercially available ascorbic acid composition including ascorbic acid in an amount of 97 wt. % based on a total weight percent of the ascorbic acid composition.

Active Agent II is a commercially available acerola powder composition including ascorbic acid in an amount of 25 wt. % based on a total weight percent of the acerola powder composition.

Active Agent III is a commercially available citrus bioflavonoid dehydrate composition.

Excipient I is a commercially available hydroxypropyl tapioca starch.

Excipient II is a commercially available potato starch.

Excipient III is a commercially available (nonmodified) tapioca starch.

Excipient IV is a commercially available pregelatinized corn starch.

Additive I is a commercially available magnesium stearate.

Additive II is a commercially available silicon dioxide.

The exemplary compositions and comparative compositions of Table I were evaluated for complete breakdown (disintegration) time of the tablet and release (dissolution) time of ascorbic acid at 2 hours, 6 hours, and 8 hours. Complete disintegration of the tablet was determined in accordance with the method described in the Revised Bulletin dated Mar. 1, 2010 entitled “<2040> Disintegration and Dissolution of Dietary Supplements” by The United States Pharmacopeial Convention (hereinafter the “Revised Bulletin”). Likewise, release (dissolution) of the ascorbic acid was determined in accordance with the method described in the Revised Bulletin. Results of the evaluation are provided in Table II below.

TABLE II
Evaluation of Compositions including Ascorbic Acid

| | Complete | Assay for | | | |
|---|---|---|---|---|---|
| | Disintegration | Ascorbic Acid | Dissolution (%) at | | |
| Composition | time (min.) | (mg/tablet) | 2 hours | 6 hours | 8 hours |
| Ex. Form. 1 | 54 | 512.2 | 36 | 67.4 | 76.7 |
| Ex. Form. 2 | ND | 719.28 | 38.2 | 76 | 77.7 |
| Ex. Form. 3 | 120 | 607.58 | 48 | 96.9 | 96.9 |
| Ex. Form. 4 | 43 | 594.66 | 52.8 | 100.3 | 100.3 |
| Comp. Form. 1 | 22 | 604.38 | 96.7 | 96.7 | 96.7 |
| Comp. Form. 2 | 37 | 622.73 | 79 | 94.9 | 94.9 |
| Comp. Form. 3 | 12.5 | 600.08 | 96.3 | 96.3 | 96.3 |
| Comp. Form. 4 | 10 | 606.32 | 100.19 | 100.19 | 100.19 |
| Comp. Form. 5 | 15 | 608.81 | 103.64 | 103.64 | 103.64 |

### Example II: Evaluation of Compositions Including Folic Acid

Exemplary and comparative compositions including folic acid (Active Agent IV) were formed as provided in Table III below. The compositions were formed into tablets using direct compression. The tablets included 0.30 to 0.55 grams of composition. The tablets had a circular configuration. Exemplary compositions (Ex. Form. 5-11) included folic acid and a modified tapioca starch in a weight ratio of from about 0.1:1 to about 4:1. Comparative compositions (Comp. Form. 6-8) included various comparative excipients or included folic acid and the excipient in a weight ratio greater than or less than 0.1:1 to about 4:1.

TABLE III
Compositions including Folic Acid

| | Ex. Form No. (wt. %) | | | | | | |
|---|---|---|---|---|---|---|---|
| Component | 5 | 6 | 7 | 8 | 9 | 10 | 11 |
| Active Agent IV | 0.21 | 0.21 | 0.19 | 0.18 | 0.17 | 0.16 | 0.18 |
| Active Agent V | 8.46 | 8.46 | 7.81 | 7.25 | 6.77 | 6.35 | 7.25 |
| Active Agent VI | 1.51 | 1.51 | 1.40 | 1.29 | 1.21 | 1.13 | 1.29 |
| Active Agent VII | 2.15 | 2.15 | 1.98 | 1.84 | 1.72 | 1.61 | 1.84 |
| Active Agent VIII | 9.4 | 9.4 | 8.67 | 8.05 | 7.52 | 7.05 | 8.05 |
| Active Agent IX | 5.42 | 5.42 | 5 | 4.64 | 4.33 | 4.06 | 4.64 |
| Excipient I | 57.02 | 57.02 | 60.33 | 63.16 | 65.62 | 67.77 | 63.16 |
| Excipient II | 0 | 0 | 0 | 0 | 0 | 0 | 0 |
| Excipient III | 0 | 0 | 0 | 0 | 0 | 0 | 0 |
| Excipient IV | 0 | 0 | 0 | 0 | 0 | 0 | 0 |
| Additive III | 0.5 | 0.5 | 0.46 | 0.43 | 0.4 | 0.38 | 0.43 |
| Additive IV | 0.33 | 0.33 | 0.31 | 0.29 | 0.27 | 0.25 | 0.29 |
| Additive V | 10 | 10 | 9.23 | 8.58 | 7.99 | 7.49 | 8.58 |
| Additive VI | 5 | 5 | 4.62 | 4.29 | 4 | 3.75 | 4.29 |
| TOTAL | 100 | 100 | 100 | 100 | 100 | 100 | 100 |

TABLE III
Compositions including Folic Acid (cont.)

| | Comp. Form. No. (wt. %) | | |
|---|---|---|---|
| Component | 6 | 7 | 8 |
| Active Agent IV | 0.18 | 0.18 | 0.18 |
| Active Agent V | 7.25 | 7.25 | 7.25 |
| Active Agent VI | 1.29 | 1.29 | 1.29 |
| Active Agent VII | 1.84 | 1.84 | 1.84 |
| Active Agent VIII | 8.05 | 8.05 | 8.05 |
| Active Agent IX | 4.64 | 4.64 | 4.64 |
| Excipient I | 0 | 0 | 0 |
| Excipient II | 63.16 | 0 | 0 |
| Excipient III | 0 | 63.16 | 0 |
| Excipient IV | 0 | 0 | 63.16 |
| Additive III | 0.43 | 0.43 | 0.43 |
| Additive IV | 0.29 | 0.29 | 0.29 |
| Additive V | 8.58 | 8.58 | 8.58 |
| Additive VI | 4.29 | 4.29 | 4.29 |
| TOTAL | 100 | 100 | 100 |

Active Agent IV is a commercially available folic acid composition including folic acid in an amount of 91.50 wt. % based on a total weight of the folic acid composition.

Active Agent V is a commercially available niacinamide composition including niacinamide in an amount of 98.50 wt. % based on a total weight of the niacinamide composition.

Active Agent VI is a commercially available thiamine mononitrate composition including thiamine mononitrate in an amount of 97.00 wt. % based on a total weight of the thiamine mononitrate composition.

Active Agent VII is a commercially available pyridoxine HCl composition including pyridoxine HCl in an amount of 98.00 wt. % based on a total weight of the pyridoxine HCl composition.

Active Agent VIII is a commercially available calcium pantothenate composition including calcium in an amount of 8.20 wt. % based on a total weight of the calcium pantothenate composition.

Active Agent IX is a commercially available biotin composition including biotin in an amount of 1.00 wt. % based on a total weight of the biotin composition.

Excipient I is a commercially available hydroxypropyl tapioca starch.

Excipient II is a commercially available potato starch.

Excipient III is a commercially available (nonmodified) tapioca starch.

Excipient IV is a commercially available pregelatinized corn starch.

Additive III is a commercially available magnesium stearate.

Additive IV is a commercially available silicon dioxide.

Additive V is a commercially available basic yeast.

Additive VI is a commercially available spirulina powder.

The exemplary compositions and comparative compositions of Table III were evaluated for complete breakdown (disintegration) time of the tablet and release (dissolution) time of folic acid at 2 hours, 4 hours, 6 hours, and 8 hours. Complete disintegration of the tablet was determined in accordance with the method described in the Revised Bulletin. Likewise, release (dissolution) of folic acid was determined in accordance with the method described in the Revised Bulletin. Results of the evaluation are provided in Table IV below.

TABLE IV
Evaluation of Compositions including Folic Acid

| | Complete | Assay for | | | | |
|---|---|---|---|---|---|---|
| | disintegration | Folic Acid | Dissolution (%) at | | | |
| Composition | time (min.) | (mg/tablet) | 2 hours | 4 hours | 6 hours | 8 hours |
| Ex. Form. 5 | 90 | 517.8 | 68.2 | 92.8 | 90.7 | 89.1 |
| Ex. Form. 6 | 53.5 | 543.4 | 40.7 | 61.7 | 80.0 | 80.1 |
| Ex. Form. 7 | 79 | 559.1 | 65.3 | 96.4 | 93.7 | 91.0 |
| Ex. Form. 8 | 77 | 575.23 | 68.6 | 83.1 | 93.8 | 88.2 |
| Ex. Form. 9 | 73 | 584.33 | 48.8 | 90.1 | 92.6 | 88.0 |
| Ex. Form. 10 | 84 | 499.73 | 67.3 | 84.4 | 97.5 | 90.8 |
| Ex. Form. 11 | 85.5 | 532.29 | 46.07 | 51.51 | 94.45 | 88.29 |
| Comp. Form. 9 | 6.5 | 565.51 | 97.85 | 93.3 | 89.93 | 80.67 |
| Comp. Form. 10 | 4 | 577 | 90.26 | 84.32 | 85.5 | 78.32 |
| Comp. Form. 11 | 14 | 548.73 | 97.17 | 92.5 | 90.54 | 85.22 |

### Example III: Evaluation of Compositions Including Caffeine

Exemplary and comparative compositions including caffeine from green tea extract (Active Agent X) were formed as provided in Table V below. The compositions were formed into tablets using direct compression. The tablets included 0.3 to 0.5 grams of composition. The tablets had a circular configuration. Exemplary compositions (Ex. Form. 12-14) included caffeine and a modified tapioca starch in a weight ratio of from about 0.1:1 to about 4:1. Comparative compositions (e.g., Comp. Form. 9) included a comparative excipient or included caffeine and the excipient in a weight ratio greater than or less than 0.1:1 to about 4:1.

TABLE V
Compositions including Caffeine

| | Ex. Form No. (wt. %) | | | |
|---|---|---|---|---|
| Component | 12 | 13 | 14 | Comp. Form. 9 (wt. %) |
| Active Agent X | 18.75 | 15.00 | 12.5 | 18.75 |
| Excipient I | 78.75 | 82 | 84.5 | 0 |
| Excipient II | 0 | 0 | 0 | 78.75 |
| Additive I | 1.5 | 2 | 2 | 1.5 |
| Additive II | 1 | 1 | 1 | 1 |
| TOTAL | 100 | 100 | 100 | 100 |

Active Agent X is a commercially available green tea extract containing caffeine.

Excipient I is a commercially available hydroxypropyl tapioca starch.

Excipient II is a commercially available microcrystalline cellulose.

Additive I is a commercially available magnesium stearate.

Additive II is a commercially available silicon dioxide.

The exemplary compositions and comparative compositions of Table V were evaluated for complete breakdown (disintegration) time of the tablet and release (dissolution) time of folic acid at 2 hours, 4 hours, 6 hours, and 8 hours. Complete disintegration of the tablet was determined in accordance with the method described in the Revised Bulletin. Likewise, release (dissolution) of caffeine was determined in accordance with the method described in the Revised Bulletin. Results of the evaluation are provided in Table VI below.

TABLE VI
Evaluation of Compositions including Caffeine

| | Complete | Assay for | | | | |
|---|---|---|---|---|---|---|
| | disintegration | Caffeine | Dissolution (%) at | | | |
| Composition | time (min.)¹ | (mg/tablet)² | 2 hours | 4 hours | 6 hours | 8 hours |
| Ex. Form. 12 | >90 | 75 | 13.14 | 22.30 | 35.09 | 43.94 |
| Ex. Form. 13 | >90 | 75 | 12.84 | 19.35 | 33.29 | 41.66 |
| Ex. Form. 14 | >90 | 75 | 15.16 | 26.00 | 34.47 | 38.53 |
| Comp. Form. 9* | 8 | 75 | 99.33 | 96.64 | 117.40 | 107.86 |

¹>90 minutes means that the evaluation exceeded the 90 minute limit of the testing equipment.
²Based on theoretical analysis.
*This did not show a controlled-release profile, as shown by the quick disintegration time of 8 minutes. Thus, dissolution profile fluctuations are to be expected, e.g., given weight variation and/or analytical variations.

### Dual Release Evaluations

In view of the findings above, where modified tapioca starch, and more so, hydroxypropyl tapioca starch, provides excellent release profiles, multilayer formulations were evaluated. Release from oral solid dosage forms like the bilayer dual release tablets in this instance, is the term used for the cumulative amount of analyte dissolved in a given amount of time. The term “dual release” generally refers to a combination of immediate-release and extended- or modified-release within the same bilayer tablet (such as that illustrated in FIGS. 1 and 2).

Dissolution profile or cumulative amount of analyte dissolved per unit time may be designed based on clinical evidence, reference peer review article and/or established monographs in an official compendium, for the analytes involved. In the absence of an established monograph, a product development team may design the dissolution profile of a desired product based on published literature and clinical evidence.

In the case of this evaluation, an immediate-release profile was designed to release not less than (“NLT”) 80%, optionally NLT 75%, of the label claim within 1 hour of in-vitro dissolution in an appropriate dissolution medium. The term “label claim” general refers to the amount of specific active (e.g. vitamin, mineral, botanical, etc.) that is listed on the product label.

Whereas an extended- or modified-release profile was designed to release NLT 65-85%, optionally NLT 80%, of the label claim within 6 hour of in-vitro dissolution in an appropriate dissolution medium. The extended-release profile was further dissected as follows:
Analytical Markers: 1. Niacinamide; and 2. Iron
   Proposed Spec. For Niacinamide:
   Cumulative % Dissolved
      At 1 Hour: not more than 40%
      At 3 Hours: 40% to 85%
      At 6 Hours: NLT 85%
   Proposed Spec. for Iron:
   Cumulative % Dissolved
      At 1 Hour: not more than 40%
      At 3 Hours: 40% to 65%
      At 6 Hours: NLT 65%

Representative analytical Markers for immediate-release and extended-release layers were selected based on the guidance stated in USP 2040 “DISINTEGRATION AND DISSOLUTION OF DIETARY SUPPLEMENTS”.

Other analytical markers include, but are not limited to, folic acid, copper, and ascorbic acid. Studying and evaluating dissolution properties of analytical markers is useful for modeling other actives (e.g., vitamins and minerals). For example, in immediate-release (IR) layer formulations, among possible vitamins and minerals added, folic acid can be used to represent the dissolution pattern of vitamins present in the IR layer, and copper can be used to represent the dissolution pattern of minerals present in the IR layer. As a specific example, one target or spec. for folic acid dissolution, is that dissolution of folic acid in 60 minutes is not less than (NLT) 80%. As another specific example, one target or spec. for copper dissolution, is that dissolution of copper in 60 minutes is NLT 80%.

Further, in extended-release (ER) layer formulations, among possible vitamins and minerals added, ascorbic acid can be used to represent the dissolution pattern of vitamins present in the layer ER, and iron can be used to represent the dissolution pattern of minerals present in the ER layer. As a specific example, targets or specs. for ascorbic acid dissolution, is that dissolution of ascorbic acid is NLT 15% in 60 minutes, is NLT 45% in 3 hours, and is NLT 80% and in 6 hours. As another specific example, targets or specs. for iron dissolution, is that dissolution of iron is NLT 15% in 60 minutes, is NLT 45% in 3 hours, and is NLT 80% in 6 hours.

### Immediate-Release Layer Formulation

The immediate-release layer was formulated to release the actives within one hour of ingestion. The formulation was similar to a traditional single layer immediate release tablets. It consisted of actives, a binder, a disintegrant, a lubricant, and a glidant. Such ingredients and amounts thereof are illustrated in Table VII further below.

### Extended-Release Layer Formulation

The extended-release layer was formulated to release the actives within 6-8 hours of ingestion. The formulation consisted of actives, a binder, a modified-release agent, a lubricant, and a glidant. Such ingredients and amounts thereof are illustrated in Table VII further below. The modified-release agent is commercially available hydroxypropyl tapioca starch. The modified-release agent and the amount and thus ratio relative to actives used, helps to achieve the unique dissolution profile as detailed above.

The modified-release agent forms an in-situ hydrophilic gel matrix once ingested. This formed gel acts as an active reservoir and the actives release from this reservoir through a variety of mechanisms. These mechanisms include, but are not limited to, actives release through a) diffusion, b) actives releasing through gel pores based on molecular size of the active, and c) gel matrix erosion over time resulting in active release.

### Example IV: Evaluation of Dual Release Bilayer Compositions Including Vitamins and Minerals

An exemplary formulation of a dual release bilayer composition including two distinct vitamin and mineral composites (Active Agents XI and XII) were formed as provided in Table VII below. The dual release bilayer composition is formed by a formulation method. The formulation method comprises the step of weighing components of each layer. The components are individually sieved and then blended together to form the compositions. The compositions are then compressed to form the layers and thus the bilayer tablet. The bilayer is then coated.

TABLE VII
Dual Release Bilayer Formulation

| No. | Ingredients | Qty. (mg) | % w/w | Purpose/Function |
|---|---|---|---|---|
| | Immediate-Release Composition/Layer | | | |
| 1 | Active Agent XI | 100.000 | 7.5188 | Active |
| 2 | Acerola Powder | 5.000 | 0.3759 | Active |
| 3 | Purple Carrot Juice Powder | 5.000 | 0.3759 | Active |
| 4 | Elderberry Water Extract | 5.000 | 0.3759 | Active |
| 5 | Centella asiatica (25:1) Extract | 120.000 | 9.0226 | Active |
| 6 | Tricalcium Phosphate, DC | 395.775 | 29.7575 | Active |
| 8 | Microcrystalline Cellulose (“MCC”) (102) | 50.625 | 3.8064 | Binder |
| 9 | Gum Mod Non-GMO | 17.000 | 1.2782 | Disintegrant |
| 10 | Magnesium Stearate, FG | 10.800 | 0.8120 | Lubricant |
| 11 | Silicon Dioxide, NF | 10.800 | 0.8120 | Glidant |
| | Extended-Release Composition/Layer | | | |
| 12 | Active Agent XII | 210.000 | 15.7895 | Active |
| 13 | Magnesium Oxide | 165.840 | 12.4692 | Active |
| 14 | Modified Tapioca Starch | 83.935 | 6.3109 | Controlled-Release |
| | | | | Agent |
| 6 | Tricalcium Phosphate, DC | 131.925 | 9.9192 | Active |
| 15 | Magnesium Stearate, FG | 9.150 | 0.6880 | Lubricant |
| 16 | Silicon Dioxide, NF | 9.150 | 0.6880 | Glidant |
| Uncoated Tablet Weight | | 1330.000 | 100.000 | |
| | Outer Coating Composition/Layer | | | |
| 17 | HPMC coating solution (solids) | 13.3000 | n/a | Film Coating |
| | | | | Agent |
| 18 | Carnauba Wax | 0.09 | n/a | Glazing Agent |
| | Coated Tablet Weight | 1343.39 | | |

Active Agent XI is a mixture of copper, iodine, molybdenum, calcium, phosphorous, boron, selenium, vitamin A, vitamin E, vitamin D, vitamin K, vitamin B12, biotin, folic acid, and riboflavin.

Active Agent XII is a mixture of zinc, chromium, manganese, iron, magnesium, vitamin C, vitamin B5, vitamin B6, thiamine, niacin (e.g. vitamin B3/niacinamide), and vitamin B12.

### Dissolution Data

Analytical markers for both layers of bilayer tablets were selected based on specified index vitamins & minerals per General Chapter USP 2040: DISINTEGRATION AND DISSOLUTION OF DIETARY SUPPLEMENTS. They were also selected based on the targeted release desired to achieve optimum release of the vitamins & minerals at their absorption sites throughout the GI tract. A mix of water-soluble vitamins and water-insoluble minerals was selected for the extended-release layer to show that this technology successfully extends the release of both.

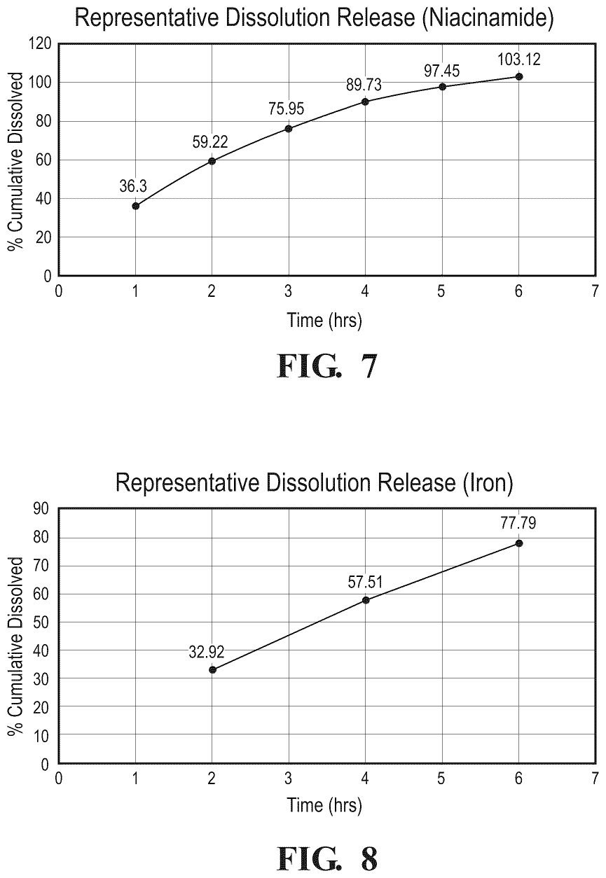

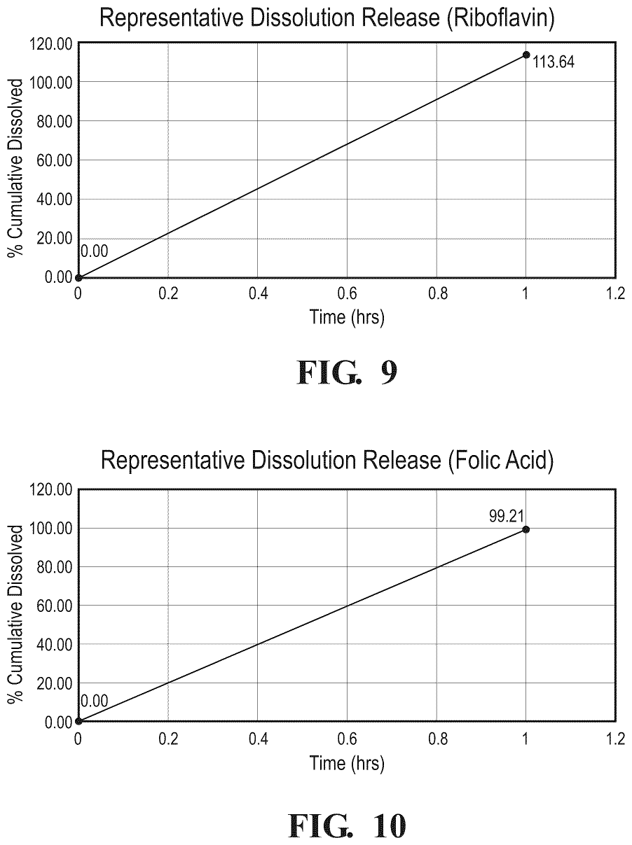

Release (dissolution) of iron (present in Active Agent XII), niacinamide, riboflavin (present in Active Agent XI), and folic acid (present in Active Agent XI) is determined in accordance with the method described in the Revised Bulletin. FIG. 7 is a graph showing the percent niacinamide release over time. FIG. 8 is a graph showing the percent iron release over time. FIG. 9 is a graph showing the percent riboflavin release over time. FIG. 10 is a graph showing the percent folic acid release over time. As illustrated, a combination of immediate- and extended-release of vitamins and minerals can be achieved.

### Further Studies

Further evaluations of dual-layer, dual-release (DLDR) formulations are conducted. Specifically, dissolution analysis was carried out on the DLDR tablets, and the selected analytes were quantified over a 6-hour period for extended-release analytes whereas a 1-hour period for the immediate release analytes. The analytes selected for extended release were Iron and Ascorbic Acid; whereas Copper and Folic Acid were tested for immediate release aspect of the DLDR tablet.

Table VIII below shows the dissolution release profile of Ascorbic Acid, Iron, Copper and Folic Acid. Please note: Time Point “0” with % cumulative dissolved “0%” was not tested. It has been added for representation of the dissolution for Copper and Folic Acid. Curves/graphs may be generated based on the values below.

TABLE VIII
Dissolution Profiles in DLDR Tablets

% Cumulative Dissolved at

| Component | 0 hrs. | 1 hr. | 3 hrs. | 6 hrs. |
|---|---|---|---|---|
| Ascorbic Acid | — | 26.82 | 57.76 | 84.68 |
| Iron | — | 26.06 | 64.7 | 91.02 |
| Copper | 0 | 83 | — | — |
| Folic Acid | 0 | 82 | — | — |

Dissolution analysis was also carried out in an Immediate release tablet with the same vitamin, mineral and botanical composition. The release of Iron and Niacinamide was studied in these tablets. Table IX below shows a comparison of Ascorbic Acid and Iron dissolution in an IR tablet vs an inventive DLDR tablet. Curves/graphs may be generated based on the values below.

TABLE IX
Dissolution Comparison

% Cumulative Dissolved at

| | 0 hrs. | 1 hr. | 3 hrs. | 6 hrs. |
|---|---|---|---|---|
| Ascorbic Acid tablet type | | | | |
| DLDR | — | 26.82 | 57.76 | 84.68 |
| IR | 0 | 112 | — | — |
| Iron tablet type | | | | |
| DLDR | — | 26.06 | 64.7 | 91.02 |
| IR | 0 | 108 | — | — |

The comparative dissolution analysis illustrated in Table IX shows the difference in release profiles of the analytes Ascorbic acid and Iron when they are formulated as an IR tablet vs a DLDR tablet. It shows that the formulation of the DLDR tablets release these analytes in a controlled manner over an extended period. The IR formulations cannot achieve the same controlled release. Another key highlight of the DLDR technology is that it controls the release of a relatively water-soluble vitamin (Niacinamide) and a relatively water-insoluble mineral (Iron) within the same dosage unit. The matrix formed in the DLDR tablets during dissolution is unique in that manner for such a release pattern.

### Further Conclusions

The results of the dissolution study show that the marker nutrients chosen for the extended-release layer have a delayed release profile where dissolution is happening within a 1 to 6 hour timeframe and the contents in this layer would be released accordingly in the GI tract. Scientific literature indicates that the nutrients in the extended-release layer have absorption sites beyond the stomach and duodenum and a delayed profile as shown in the studies herein would help these nutrients to be more accessible to jejunum and subsequent sites where they are known to be absorbed.

In contrast, the ingredient added in the conventional IR layer are expected to be released within an hour as indicated by the dissolution profile of the two reference nutrients and thus as literature indicates, would be accessible to the stomach, duodenum and/or jejunum as those would be their desired sites of absorption.

This novel methodology of ‘Dual Layer Dual Release’ combines the beneficial mechanisms of bilayer technology (having both immediate and extended-release components) which may help in the better accessibility (and their possible availability) of the nutrients in the present formulation.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, different, special, and/or unexpected results may be obtained from each member of the respective Markush group independent from all other Markush members. Each member of a Markush group may be relied upon individually and or in combination and provides adequate support for specific embodiments within the scope of the appended claims.

Further, any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present invention has been described herein in an illustrative manner, and it is to be understood that the terminology which has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both single and multiple dependent, is herein expressly contemplated.

1. A multilayer formulation for controlled-release and immediate-release of active agents after administration of the multilayer formulation to a subject, the multilayer formulation comprising:
i) a first layer comprising a controlled-release composition;
ii) a second layer disposed adjacent the first layer and comprising an immediate-release composition different from the controlled-release composition; and
iii) optionally, an outer coating covering the first and second layers;
wherein the controlled-release composition of the first layer comprises:
a first active agent; and
a controlled-release excipient comprising a modified tapioca starch;
wherein the controlled-release composition is adapted to release;
no greater than 50 wt. % of the first active agent within 1 hour of ingestion of the multilayer formulation by the subject, and/or
not less than 55 wt. % of the first active agent within 6 hours after ingestion of the multilayer formulation by the subject,
each based on a total weight of the first active agent present in the controlled-release composition; and
wherein the immediate-release composition of the second layer comprises:
a second active agent; and
an excipient different from the controlled-release excipient;
wherein the immediate-release composition is adapted to release;
not less than 60 wt. % of the second active agent within 1 hour after ingestion of the multilayer formulation by the subject, based on a total weight of the second active agent present in the immediate-release composition.

2. The multilayer formulation according to claim 1, wherein the weight ratio of first active agent and controlled-release excipient in the first layer is from about 0.001:1 to about 4:1, or optionally from about 0.4:1 to about 3.5:1.

3. The multilayer formulation according to claim 1, wherein:
i) the controlled-release excipient is the modified tapioca starch; and/or
ii) upon administration to the subject, the modified tapioca starch contributes to controlled-release of the first active agent from the first layer; and/or
iii) the modified tapioca starch is the only excipient present in the first layer.

4. The multilayer formulation according to claim 1, wherein the modified tapioca starch comprises or is a hydroxypropyl tapioca starch.

5. The multilayer formulation according to claim 1, wherein at least the first layer is, or optionally both the first and second layers are, substantially or completely free of: i) polymeric crosslinking; and/or ii) hydroxypropyl methylcellulose starch (HPMC); and/or iii) high-amylose starch (HAS); and/or iv) methylcellulose (MC); and/or v) hydroxypropyl cellulose (HPC); and/or vi) ethyl cellulose (EC).

6. The multilayer formulation according to claim 1, wherein the second layer provides immediate-release of the second active agent at a first location in the gastrointestinal (GI) tract of the subject, optionally wherein the first location in the GI tract of the subject is selected from the group consisting of the stomach, duodenum, and combinations thereof.

7. The multilayer formulation according to claim 1, wherein the immediate-release composition is adapted to release not less than 65 wt. %, or optionally not less than 70 wt. %, or optionally not less than 75 wt. %, of the second active agent within 1 hour after ingestion of the multilayer formulation by the subject, based on a total weight of the second active agent present in the immediate-release composition.

8. The multilayer formulation according to claim 1, wherein the first layer provides controlled-release of the first active agent at a second location in the gastrointestinal (GI) tract of the subject, optionally wherein the second location in the GI tract of the subject is selected from the group consisting of the duodenum, jejunum, ileum, large intestine, and combinations thereof.

9. The multilayer formulation according to claim 1, wherein the controlled-release composition is adapted to release:
i) no greater than 45 wt. %, or optionally no greater than 40 wt. %, of the first active agent within 1 hour of ingestion of the multilayer formulation by the subject; and/or
ii) not less than 60 wt. %, or optionally not less than 65 wt. %, of the first active agent within 6 hours after ingestion of the multilayer formulation by the subject,
each based on a total weight of the first active agent present in the controlled-release composition.

10. The multilayer formulation according to claim 1, wherein each of the active agents is individually selected from the group consisting of vitamins, minerals, botanicals, phytochemicals, and combinations thereof.

11. The multilayer formulation according to claim 1, wherein each of the active agents is individually, or optionally wherein at least the first active agent is, selected from the group consisting of zinc, chromium, manganese, iron, magnesium, calcium, vitamin C, vitamin B5, vitamin B6, thiamine, niacin, vitamin B12, and combinations thereof.

12. The multilayer formulation according to claim 1, wherein each of the active agents is individually, or optionally wherein at least the second active agent is, selected from the group consisting of copper, iodine, molybdenum, calcium, phosphorous, boron, selenium, vitamin A, vitamin E, vitamin D, vitamin K, vitamin B12, biotin, folic acid, riboflavin, and combinations thereof.

13. The multilayer formulation according to claim 1, wherein at least one of the compositions comprises at least one component selected from the group consisting of binders, lubricants, glidants, and combinations thereof, and optionally wherein the immediate-release composition comprises at least one disintegrant.

14. The multilayer formulation according to claim 1, wherein the multilayer formulation is in the form of a tablet, and optionally wherein the tablet is a bilayer tablet.

15. The multilayer formulation according to claim 14, wherein the first and second layers each comprise abutting substantially planar layers which form the tablet.

16. The multilayer formulation according to claim 1, wherein the outer coating is present and covers the first and second layers.

17. A method of manufacturing the multilayer formulation according to claim 1, the method comprising:
providing the controlled-release composition;
providing the immediate-release composition;
forming the first layer from the controlled-release composition;
forming the second layer from the immediate-release composition; and
combining the first and second layers to form the multilayer formulation.

18. The method according to claim 17, further comprising coating the first and second layers with a coating composition to form the outer coating covering the first and second layers.

19. A method of delivering active agents to a subject, the method comprising:
providing a multilayer formulation; and
administering the multilayer formulation to the subject;
wherein the multilayer formulation is according to claim 1.

20. The multilayer formulation according to claim 1, wherein:
i) the modified tapioca starch comprises or is a hydroxypropyl tapioca starch;
ii) at least the first layer is, or optionally both the first and second layers are, substantially or completely free of: i) polymeric crosslinking; and/or ii) hydroxypropyl methylcellulose starch (HPMC); and/or iii) high-amylose starch (HAS); and/or iv) methylcellulose (MC); and/or v) hydroxypropyl cellulose (HPC); and/or vi) ethyl cellulose (EC);
iii) the first active agent is selected from the group consisting of zinc, chromium, manganese, iron, magnesium, calcium, vitamin C, vitamin B5, vitamin B6, thiamine, niacin, vitamin B12, and combinations thereof;
iv) the second active agent is selected from the group consisting of copper, iodine, molybdenum, calcium, phosphorous, boron, selenium, vitamin A, vitamin E, vitamin D, vitamin K, vitamin B12, biotin, folic acid, riboflavin, and combinations thereof; and
v) the multilayer formulation is in the form of a tablet, and optionally wherein the tablet is a bilayer tablet.
